SEQUENCE LISTING (99 sequences)

SEQ ID NO: 1 - DNA, 3217 bp, Petunia hybrida
gttctgattttgaggatatttgcatgcatgcatgcactacttgtaacgctagttgtagttgctaagggaattaaggtacattgatttggtcaatttttttgttcacttggattatcaaaggcgcctcaagcagctacattatcctattcatcactaaacatggccgaagatctggagagacctttactaggtcctgataatttcagtcgggaagggattgatctggaaaagttgccactcgaacaagtttttgaagaattaagaacatcaaaggaagggctttcagatgaagatgcagaggagcgactgaacatattcgggccaaacaagcttgaagagaagcgagagaacaagtttattaagtttcttggcttcatgtggaatcctttgtcttgggtgatggaagcagccgcaatcatggcaattgctctagctaatggtggaggacaaggtcctgactggcaggactttgtaggcattgtttgtctgttgttgataaattcaacaattagctttatagaggaaaataatgctgggaatgctgcagcagctctcatggcacgtttagctcctagaactaaggtccttagagatgggaggtggcaagaaaaagatgcagctattctagtgccaggagacatcattagtataaagcttggtgatatcatccctgcagatgcccgcttgcttgaaggggatcccttgaaagtagatcagtcagctcttactggggaatccttacccgtcacgaaaaagacaggagatgaagttttctctggatctacttgtaaacatggagaaattgaagcagtagtaattgccactggagttcactctttctttggaaaagctgcacatcttgttgactccactcaagtcactggtcacttccagaaggtccttgcttctattggaaatttctgcatatgctcaatagcaatgggaatgatacttgaaatcattgtcatgttccctgttcagaatcgttcatataggactggaattaacaacctccttgttctcttaattggtggaataccaatagctatgccaacagtcctatcagttactcttgctattggctctcatcgactctctcaacagggtgctattacaaaaaggatgactgccattgaagagatggctggtatggatgtactctgcagtgacaagacagggacgctaaccctgaatcgcctcactattgataggaatcttattgaggtgttccaaaaagatatggacaaagatatggttgtgttacttgctgccagagcatcaagactggaaaatcaggatgctattgatgcagcagttatcaacatgcttgctgatccaaaggaggcacgtgcaaatatcagggaagtgcatttccttccattcaatcctgtcgacaaacggactgcaatcacttacattgattcagatggaaagtggtatcgagcaagcaaaggagctcctgaacagattctgaccttgtgccaagagaagcaacagatagctgcaaaagtgcacacgatcattgacaagtttgctgaaagagggttacgatctcttgctgtttcctttcaggaaattccagagaactcaaaggagagtcctggagggccttggcaattttgtggattgcttcccttgtttgatccaccaaggcacgatagtgctgagaccattcggagagcactcaacttaggagtttgtgttaagatgattactggtgaccagttggccattgcaaaggaaacaggccgacgacttggcatgggaacaaatatgtatccctcatgttcgttgtttggtcgtgacaaggatgaaactgaagctctaccagttgatgagctcattgaaaaagcagatggctttgctggtgtatttcctgagcacaaatatgagatagtaaaaatcctgcaaatgaatgagcacgtggttggaatgactggtgatggagtaaatgatgcaccagctctcaagaaagcggatattggtatagcagtcgctgatgctacagatgctgctaggagtgctgctgatcttgtcttgactgagcctggcttaagtgtgattgtcagtgctgttttgactagcagggctatatttcaaagaatgaaaattatacaattcatgcttctggcgctaatatggaaatatgactttcctccattcatggtactgataatagcaatcctaaatgatggcactattatgactatttcaaaagatcgggttaaaccatctccaagacctgatagttggaagcttaacgagatatttgcaactggcgttgtgcttggcacatatcttgctttagttactgtgttgttttactggcttgcagacagcactcaattctttgaggctcacttccatgtaaaatctctaagtggaagtagtgaagaaatgtcatcagccgtttatctgcaagtaagtattatcagccaggcgctaatatttgttacccgcagtcagagttggtcatttacagagagaccgggggctcttttgatgtttgcatttgtggtggcacaactggttgctaccttgatagcagtttatgcacatattagcttcgcatcagttagaggcatcggttggggatgggcaggtgtcatatggttatacagtcttattttctatattcccttggatataatcaaatttgctgtttgttatgctctgactggagaagcctggaacctacttttcgataagaagactgcttttacgtcgaaaaaggactatggaagggaggacagagaggcccagtgggtgctttctcagagaagtttacagcgggtaatatcaccagagtttgaacctagaagcaggaggccatctatgattgctgaacaagctaagcgtcgagccgaaataaccaggcttagagagttgtacactttgagaggtcatatagaatcagtagcaagactcaagaatctggaccttaacaaaattcaaacagctcatacagtttgatatttgaatctgcatgaaatctcatctcctcctataacaaacttctggctccatgttgtcttttaagtaactttctatcatcatgtggtccatgagagacatgtcaagaggcaatgttgttcatgatcttctgccaaaaaaaccatgttgaggatgtttctttgaagctggacacctcatatcattcattaatttctcccggggaagtgaatctaggacattgtatcatgcttttaaaaaaaaa

SEQ ID NO: 2 - protein, 937 aa, Petunia hybrida
MAEDLERPLLGPDNFSREGIDLEKLPLEQVFEELRTSKEGLSDEDAEERLNIFGPNKLEEKRENKFIKFLGFMWNPLSWVMEAAAIMAIALANGGGQGPDWQDFVGIVCLLLINSTISFIEENNAGNAAAALMARLAPRTKVLRDGRWQEKDAAILVPGDIISIKLGDIIPADARLLEGDPLKVDQSALTGESLPVTKKTGDEVFSGSTCKHGEIEAVVIATGVHSFFGKAAHLVDSTQVTGHFQKVLASIGNFCICSIAMGMILEIIVMFPVQNRSYRTGINNLLVLLIGGIPIAMPTVLSVTLAIGSHRLSQQGAITKRMTAIEEMAGMDVLCSDKTGTLTLNRLTIDRNLIEVFQKDMDKDMVVLLAARASRLENQDAIDAAVINMLAESKEARANIREVHFLPFNPVDKRTAITYIDSDGKWYRASKGAPEQILTLCQEKQQIAAKVHTIIDKFAERGLRSLAVSFQEIPENSKESPGGPWQFCGLLPLFDPPRHDSAETIRRALNLGVCVKMITGDQLAIAKETGRRLGMGTNMYPSCSLFGRDKDETEALPVDELIEKADGFAGVFPEHKYEIVKILQMNEHVVGMTGDGVNDAPALKKADIGIAVADATDAARSAADLVLTEPGLSVIVSAVLTSRAIFQRMKIIQFMLLALIWKYDFPPFMVLIIAILNDGTIMTISKDRVKPSPRPDSWKLNEIFATGVVLGTYLALVTVLFYWLADSTQFFEAHLHVKSLSGSSEEMSSAVYLQVSIISQALIFVTRSQSWSFTERPGALLMFAFVVAQLVATLIAVYAHISFASVRGIGWGWAGVIWLYSLIFYIPLDIIKFAVCYALTGEAWNLLFDKKTAFTSKKDYGREDREAQWVLSQRSLQRVISPEFEPRSRRPSMIAEQAKRRAEITRLRELYTLRGHIESVARLKNLDLNKIQTAHTV

SEQ ID NO: 3 - DNA, 687 bp, Petunia hybrida
  misc_feature 9: n = A,T,C or G
ccttttcgncgcgccgtccaccaccaccaacatcaagattccttcttaagcaggaaactaccagttgagcctatggctgcaccaagcctaacaaaacaggagattgacaagtattggaagcagaagcgcatgactgaagaagaacattttcttgatgcaattaaggctgcagccagaatcagggcacgcaatctctcggaggaggactacacacacttcgtggattctttgaaggaggatggtgattataaggagaatggggtaactaagaacatctccaaaattgatgaggacatgaaggagcaaagagttggcataaaagactggtggacaaagagcaaatatgcttatctaaaccagccagcagtgaaatcgatggaaggcaaaggctcctcttatattccccaattatattgctacaaggctcctcctccaccagttgcaaccacttttggcatattctagggaattttcatatatacactcatattcatgtattcgtattgtgcttgtagtatataattgaattgttaaagtgtgcatcctccaaagaattgtctcatatgattgaaattgttgtaagtattgcgcttgcgaactactttggttttatgaaaccatgtgttaataaataatataatggttctattatattgttggatgcttgatataaaaaaaaaaaaaaaa

SEQ ID NO: 4 - protein, 154 aa, Petunia hybrida
PFRRAVHHHQHQDSFLSRKLPVEPMAAPSLTKQEIDKYWKQKRMTEEEHFLDAIKAAARIRARNLSEEDYTHFVDSLKEDGDYKENGVTKNISKIDEDMKEQRVGIKDWWTKSKYAYLNQPAVKSMEGKGSSYIPQLYCYKAPPPPVATTFGIF

SEQ ID NO: 5 - DNA, 1169 bp, Petunia hybrida
  misc_feature 131, 146, 149: n = A,T,C or G
ggcacgagtctgcttttgaatgggtgatcaacaatggtggcattgattcagcttctgattatccctatactgcaattcaaggcacttgcaatatcaccaaggagaagactaagattgttacaatgatgganaccaagatgttgcanaanaggaaagtgccctctctcttgtgctgctgcgcaacaacctgttatgttggcataatggctctagcttgggatttccaactatatacagggggaatttatgatggagattgtgtctacaatccaaatgatgtgcgaccatgcagtataatagtaggatatggttctgaaggaatgaccaatattggattatcaagaattcatggggtacatcttggggaatggagggtatgcatatataaaaaggaacactgatttgtcatatggtgtttgtgccattaactcaattgcttcatatccaacgaaagaatcgtcttcttcgtcgcctccttatccatctccagcccttcccccacctccaccgccttctccatcaccaagtgaatgtggagactactatttctgttcaataggccaaacatgttgctgtgagttagagttgtttggagtctgcttcattcagggttgctgtccttatgaaaatggtgtttgctgtgacaaatcagaatactgctgcccaagtggctattctgtttgttctgttaatcaaggcatgtgcctcaaggagtatggcgactatcttggtggttgccagcaaagaagagaagaatagccaagtacaagttatcatggagtactagtacaaaattaacaatggagatggaccaacatctgcagtggaagaggaatgaatttttagaaatgctgaaggtacagttgatactaaagaagactcttagcattgtcaaatggtttccaggtactaaaggctaggacatatagatggttcttgcttatgaaaccatgaataattacttcatgaatactttgcttgtttctaattgatagtactattttcttgatattaaggattgaatagtgagttctgtgacacctttaatgttcttagtggctcctcaaagtagaactctacaaagtgaattcgtaaatgtaataatgtatatggatgctgtttgaatgtcattacatttaggttgttttgtgaaaaaaaaaaaaaaaaaa

SEQ ID NO: 6 - protein, 130 aa, Petunia hybrida
MAAPSLTKQEIDKYWKQKRMTEEEHFLDAIKAAARIRARNLSEEDYTHFVDSLKEDGDYKENGVTKNISKIDEDMKEQRVGIKDWWTKSKYAYLNQPAVKSMEGKGSSYIPQLYCYKAPPPPVATTFGIF

SEQ ID NO: 7 - DNA, 16 bp, Artificial Sequence
  Primer
gacgatgagtcctgag

SEQ ID NO: 8 - DNA, 14 bp, Artificial Sequence
  Primer
tactcaggactcat

SEQ ID NO: 9 - DNA, 19 bp, Artificial Sequence
  Primer
gacgatgagtcctgagtaa

SEQ ID NO: 10 - DNA, 20 bp, Artificial Sequence
  Primer
gacgatgagtcctgagtaaa

SEQ ID NO: 11 - DNA, 20 bp, Artificial Sequence
  Primer
gacgatgagtcctgagtaac

SEQ ID NO: 12 - DNA, 20 bp, Artificial Sequence
  Primer
gacgatgagtcctgagtaag

SEQ ID NO: 13 - DNA, 20 bp, Artificial Sequence
  Primer
gacgatgagtcctgagtaat

SEQ ID NO: 14 - DNA, 19 bp, Artificial Sequence
  Primer
gtgatatctccactgacgt

SEQ ID NO: 15 - DNA, 17 bp, Artificial Sequence
  Primer
ctcgtagactgcgtacc

SEQ ID NO: 16 - DNA, 15 bp, Artificial Sequence
  Primer
aattggtacgcagtc

SEQ ID NO: 17 - DNA, 18 bp, Artificial Sequence
  Primer
agactgcgtaccaattca

SEQ ID NO: 18 - DNA, 18 bp, Artificial Sequence
  Primer
agactgcgtaccaattcc

SEQ ID NO: 19 - DNA, 18 bp, Artificial Sequence
  Primer
agactgcgtaccaattcg

SEQ ID NO: 20 - DNA, 18 bp, Artificial Sequence
  Primer
gatgagtcctgagtaaaa

SEQ ID NO: 21 - DNA, 18 bp, Artificial Sequence
  Primer
gatgagtcctgagtaaac

SEQ ID NO: 22 - DNA, 18 bp, Artificial Sequence
  Primer
gatgagtcctgagtaaag

SEQ ID NO: 23 - DNA, 18 bp, Artificial Sequence
  Primer
gatgagtcctgagtaaat

SEQ ID NO: 24 - DNA, 18 bp, Artificial Sequence
  Primer
gatgagtcctgagtaaca

SEQ ID NO: 25 - DNA, 18 bp, Artificial Sequence
  Primer
gatgagtcctgagtaacc

SEQ ID NO: 26 - DNA, 18 bp, Artificial Sequence
  Primer
gatgagtcctgagtaacg

SEQ ID NO: 27 - DNA, 18 bp, Artificial Sequence
  Primer
gatgagtcctgagtaact

SEQ ID NO: 28 - DNA, 18 bp, Artificial Sequence
  Primer
gatgagtcctgagtaaga

SEQ ID NO: 29 - DNA, 18 bp, Artificial Sequence
  Primer
gatgagtcctgagtaagc

SEQ ID NO: 30 - DNA, 18 bp, Artificial Sequence
  Primer
gatgagtcctgagtaagg

SEQ ID NO: 31 - DNA, 18 bp, Artificial Sequence
  Primer
gatgagtcctgagtaagt

SEQ ID NO: 32 - DNA, 18 bp, Artificial Sequence
  Primer
gatgagtcctgagtaata

SEQ ID NO: 33 - DNA, 18 bp, Artificial Sequence
  Primer
gatgagtcctgagtaatc

SEQ ID NO: 34 - DNA, 18 bp, Artificial Sequence
  Primer
gatgagtcctgagtaatg

SEQ ID NO: 35 - DNA, 18 bp, Artificial Sequence
  Primer
gatgagtcctgagtaatt

SEQ ID NO: 36 - DNA, 18 bp, Artificial Sequence
  Primer
gactgcgtaccaattcaa

SEQ ID NO: 37 - DNA, 18 bp, Artificial Sequence
  Primer
gactgcgtaccaattcac

SEQ ID NO: 38 - DNA, 18 bp, Artificial Sequence
  Primer
gactgcgtaccaattcag

SEQ ID NO: 39 - DNA, 18 bp, Artificial Sequence
  Primer
gactgcgtaccaattcat

SEQ ID NO: 40 - DNA, 18 bp, Artificial Sequence
  Primer
gactgcgtaccaattcca

SEQ ID NO: 41 - DNA, 18 bp, Artificial Sequence
  Primer
gactgcgtaccaattccc

SEQ ID NO: 42 - DNA, 18 bp, Artificial Sequence
  Primer
gactgcgtaccaattccg

SEQ ID NO: 43 - DNA, 18 bp, Artificial Sequence
  Primer
gactgcgtaccaattcct

SEQ ID NO: 44 - DNA, 18 bp, Artificial Sequence
  Primer
gactgcgtaccaattcga

SEQ ID NO: 45 - DNA, 18 bp, Artificial Sequence
  Primer
gactgcgtaccaattcgc

SEQ ID NO: 46 - DNA, 18 bp, Artificial Sequence
  Primer
gactgcgtaccaattcgg

SEQ ID NO: 47 - DNA, 18 bp, Artificial Sequence
  Primer
gactgcgtaccaattcgt

SEQ ID NO: 48 - DNA, 18 bp, Artificial Sequence
  Primer
gactgcgtaccaattcta

SEQ ID NO: 49 - DNA, 18 bp, Artificial Sequence
  Primer
gactgcgtaccaattctc

SEQ ID NO: 50 - DNA, 18 bp, Artificial Sequence
  Primer
gactgcgtaccaattctg

SEQ ID NO: 51 - DNA, 18 bp, Artificial Sequence
  Primer
gactgcgtaccaattctt

SEQ ID NO: 52 - DNA, 23 bp, Artificial Sequence
  Primer
ggaccttaacaaaattcaaacag

SEQ ID NO: 53 - DNA, 21 bp, Artificial Sequence
  Primer
aaattaatgaatgatatgagg

SEQ ID NO: 54 - DNA, 20 bp, Artificial Sequence
  Primer
tgaagaaatgtcatcagccg

SEQ ID NO: 55 - DNA, 20 bp, Artificial Sequence
  Primer
gttcagcaatcatagatggc

SEQ ID NO: 56 - DNA, 21 bp, Artificial Sequence
  Primer
gctctgactggagaagcctgg

SEQ ID NO: 57 - DNA, 26 bp, Artificial Sequence
  Primer
ccaagagaagcaacagatagctgcaa

SEQ ID NO: 58 - DNA, 26 bp, Artificial Sequence
  Primer
ttgcagctatctgttgcttctcttgg

SEQ ID NO: 59 - DNA, 26 bp, Artificial Sequence
  Primer
gaatcaatgtaagtgattgcagtccg

SEQ ID NO: 60 - DNA, 24 bp, Artificial Sequence
  Primer
aactgataggactgttggcatagc

SEQ ID NO: 61 - DNA, 23 bp, Artificial Sequence
  Primer
gctggtgcatcatttactccatc

SEQ ID NO: 62 - DNA, 23 bp, Artificial Sequence
  Primer
atggccgaagatctggagagacc

SEQ ID NO: 63 - DNA, 23 bp, Artificial Sequence
  Primer
ctgcagggatgatatcaccaagc

SEQ ID NO: 64 - DNA, 26 bp, Artificial Sequence
  Primer
ctgataatagcaatcctaaatgatgg

SEQ ID NO: 65 - DNA, 36 bp, Artificial Sequence
  Primer
cggaattcatggccgaagatctggagagacctttac

SEQ ID NO: 66 - DNA, 36 bp, Artificial Sequence
  Primer
cccgggcttctccagtcagagcatatcaaacagcaa

SEQ ID NO: 67 - DNA, 31 bp, Artificial Sequence
  Primer
aagaattcgtttgttatgctctgactggaga

SEQ ID NO: 68 - DNA, 24 bp, Artificial Sequence
  Primer
gactgcgggtaacaaatattagcg

SEQ ID NO: 69 - DNA, 25 bp, Artificial Sequence
  Primer
gcaaatatcagggaagtgcatttcc

SEQ ID NO: 70 - DNA, 37 bp, Artificial Sequence
  Primer
cggaattctcgcaaatatcagggaagtgcatttcctt

SEQ ID NO: 71 - DNA, 30 bp, Artificial Sequence
  Primer
ttatgaatcaatgtaagtgattgcagtccg

SEQ ID NO: 72 - DNA, 29 bp, Artificial Sequence
  Primer
tagcccatggccgaagatctggagagacc

SEQ ID NO: 73 - DNA, 33 bp, Artificial Sequence
  Primer
catgagccatggacaaactgtatgagctgtttg

SEQ ID NO: 74 - DNA, 25 bp, Artificial Sequence
  Primer
gcttgctgatccaaaggaggcacgt

SEQ ID NO: 75 - DNA, 22 bp, Artificial Sequence
  Primer
gtaaggattccccagtaagagc

SEQ ID NO: 76 - DNA, 34 bp, Artificial Sequence
  Primer
cgggatcctggagccagaagtttgttataggagg

SEQ ID NO: 77 - DNA, 22 bp, Artificial Sequence
  Primer
ggtcttggagatggtttaaccc

SEQ ID NO: 78 - DNA, 25 bp, Artificial Sequence
  Primer
gctgctaggagtgctgctgatcttg

SEQ ID NO: 79 - DNA, 28 bp, Artificial Sequence
  Primer
gcatgatacaatgtcctagattcacttc

SEQ ID NO: 80 - DNA, 30 bp, Artificial Sequence
  Primer
ctaaccatggccgaagacctggagagacct

SEQ ID NO: 81 - DNA, 45 bp, Artificial Sequence
  Primer
gtttgatcagacgtcacatgtctccaaactgtatgagctgtttga

SEQ ID NO: 82 - DNA, 22 bp, Artificial Sequence
  Primer
cttgttgacagcaccaacaatg

SEQ ID NO: 83 - DNA, 25 bp, Artificial Sequence
  Primer
caaggatctatcgacactcaacttg

SEQ ID NO: 84 - DNA, 21 bp, Artificial Sequence
  Primer
gttcgcaagcgcaatacttac

SEQ ID NO: 85 - DNA, 20 bp, Artificial Sequence
  Primer
ggaattcggcacgaggtcac

SEQ ID NO: 86 - DNA, 18 bp, Artificial Sequence
  Primer
aagagtagctgatcatgg

SEQ ID NO: 87 - DNA, 25 bp, Artificial Sequence
  Primer
gatgaggacatgaaggagcaaagag

SEQ ID NO: 88 - DNA, 24 bp, Artificial Sequence
  Primer
cttcagtcttgcgtttctgcttcc

SEQ ID NO: 89 - DNA, 24 bp, Artificial Sequence
  Primer
ctcctgttttgtcaggcttggtgc

SEQ ID NO: 90 - DNA, 22 bp, Artificial Sequence
  Primer
cggcggcggtggacttgtcttc

SEQ ID NO: 91 - DNA, 35 bp, Artificial Sequence
  Primer
gctctagactagaatatgccaaaagtggttgcaac

SEQ ID NO: 92 - DNA, 36 bp, Artificial Sequence
  Primer
atcgaattcatggctgcaccaagcctaacaaaacag

SEQ ID NO: 93 - DNA, 37 bp, Artificial Sequence
  Primer
accgctcgagctagaatatgccaaaagtggttgcaac

SEQ ID NO: 94 - DNA, 21 bp, Artificial Sequence
  Primer
cctgtatatagttggaaatcc

SEQ ID NO: 95 - DNA, 21 bp, Artificial Sequence
  Primer
caaggcacttgcaatatcacc

SEQ ID NO: 96 - DNA, 23 bp, Artificial Sequence
  Primer
gtaatgacattcaaacagcatcc

SEQ ID NO: 97 - DNA, 25 bp, Artificial Sequence
  Primer
cttcgtcgcctccttatccatctcc

SEQ ID NO: 98 - DNA, 22 bp, Artificial Sequence
  Primer
ggattatcaagaattcatgggg

SEQ ID NO: 99 - DNA, 23 bp, Artificial Sequence
  Primer
gcctccttatccatctccagccc

(19) United States
(12) Patent Application Publication
(10) Pub. No.: US 2009/0217413 A1
(43) Pub. Date: Aug. 27, 2009

(54) PLANT GENETIC SEQUENCES ASSOCIATED WITH VACUOLAR PH AND USES THEREOF

(75) Inventors: Francesca Quattrocchio, Amsterdam (NL); Ronald Koes, Amsterdam (NL); Walker Verweij, Almere Buiten (NL); Keez Spelt, Amsterdam (NL)

Correspondence Address: KNOBBE MARTENS OLSON & BEAR LLP, 2040 MAIN STREET, FOURTEENTH FLOOR, IRVINE, CA, 92614, US

(73) Assignees: VERENIGING VOOR CHRISTELIJK HOGER ONDERWIJS WETENSCHAPPELIJK ONDERZOEK EN PATIENTENZORG, Amesterdam (NL); INTERNATIONAL FLOWER DEVELOPEMENT PTY LTD., Bundoora (AU)

(21) Appl. No.: 11/910,732

(22) PCT Filed: Apr. 4, 2006
(86) PCT No.: PCT/AU06/00451

§ 371 Date: Jun. 25, 2008

(30) Foreign Application Priority Data
Apr. 4, 2005 (AU) 2005901631

(51) Int. Cl. A01H 1/00; C07H 21/04; C12N 15/63; C12N 5/10; A01H 5/00

(52) U.S. Cl. 800/278; 536/23.1; 435/320.1; 435/419; 800/298

(57) ABSTRACT

The present invention relates generally to the field of plant molecular biology and agents useful in the manipulation of plant physiological or biochemical properties. More particularly, the present invention provides genetic and proteinaceous agents capable of modulating or altering the level of acidity or alkalinity in a cell, group of cells, organelle, part or reproductive portion of a plant. Even more particularly, the present invention contemplates methods and agents for modulating or altering pH levels in the vacuole of a cell, group of cells, organelle, part or reproductive portion of a plant. The present invention further provides genetically altered plants, plant parts, progeny, subsequent generations and reproductive material including flowers or flowering parts having cells exhibiting an altered vacuolar pH compared to a non-genetically altered plant. The present invention still further provides methods for modulating or altering flower color in a plant. Even more particularly, the present invention provides for down regulation of pH in a plant which results in a bluer color in the plant; especially in the flower

### BACKGROUND OF THE INVENTION

1. Field of the Invention

The present invention relates generally to the field of plant molecular biology and agents useful in the manipulation of plant physiological or biochemical properties. More particularly, the present invention provides genetic and proteinaceous agents capable of modulating or altering the level of acidity or alkalinity in a cell, group of cells, organelle, part or reproductive portion of a plant. Even more particularly, the present invention contemplates methods and agents for modulating or altering pH levels in the vacuole of a cell, group of cells, organelle, part or reproductive portion of a plant. The present invention further provides genetically altered plants, plant parts, progeny, subsequent generations and reproductive material including flowers or flowering parts having cells exhibiting an altered vacuolar pH compared to a non-genetically altered plant. The present invention still further provides methods for modulating or altering flower color in a plant. Even more particularly, the present invention provides for down regulation of pH in a plant which results in a bluer color in the plant; especially in the flower

2. Description of Prior Art

Reference to any prior art in this specification is not, and should not be taken as, an acknowledgment or any form of suggestion that this prior art forms part of the common general knowledge in any country.

Bibliographic details of references provided in the subject specification are listed at the end of the specification.

The cut-flower, ornamental and agricultural plant industries strive to develop new and different varieties of plants with features such as novel flower colors, better taste/flavour of fruits (e.g. grapes, apples, lemons, oranges) and berries (e.g. strawberries, blueberries), improved yield, longer life, more nutritious, novel colored seeds for use as proprietary tags, etc.

Furthermore, plant byproduct industries which utilize plant parts value novel products which have the potential to impart altered characteristics to their products (e.g. juices, wine) such as, appearance, style, taste, smell and texture.

In the cut flower and ornamental plant industries, an effective way to create such novel varieties is through the manipulation of flower color. Classical breeding techniques have been used with some success to produce a wide range of colors for almost all of the commercial varieties of flowers and/or plants available today. This approach has been limited, however, by the constraints of a particular species' gene pool and for this reason it is rare for a single species to have the full spectrum of colored varieties. For example, the development of novel colored varieties of plants or plant parts such as flowers, foliage and stems would offer a significant opportunity in both the cut flower and ornamental markets. In the cut flower or ornamental plant industry, the development of novel colored varieties of major flowering species such as rose, chrysanthemum, tulip, lily, carnation, gerbera, orchid, lisianthus, begonia, torenia, geranium, petunia, nierembergia, pelargonium, iris, impatiens and cyclamen would be of great interest. A more specific example would be the development of a blue rose for the cut flower market.

To date, creation of a “true” blue shade in cut flowers has proven to be extremely difficult. Success in creating colors in the “blue” range has provided a series of purple colored carnation flowers (see the website for FlorigenePty Ltd, Melbourne, Australia) (International Patent Application PCT/AU96/00296). These are now on the market in several countries around the world. There is a need, however, to generate altered flower colors in other species in addition to bluer colors in carnation and other cut flower species such as rose, gerbera and chrysanthemum. It is apparent that other plants have been recalcitrant to genetic manipulation of flower color due to certain physiological characteristics of the cells. One such physiological characteristics is vacuolar pH.

In all living cells, the pH of the cytoplasm is about neutral, whereas in the vacuoles and lysosomes an acidic environment is maintained. The H⁺-gradient across the vacuolar membrane is a driving force that enables various antiporters and symporters to transport compounds across the vacuolar membrane. The acidification of the vacuolar lumen is an active process. Physiological work indicated that two proton pumps, a vacuolar H⁺ pumping ATPase (vATPase) and a vacuolar pyrophosphatase (V-PPase), are involved in vacuolar acidification.

Vacuoles have many different functions and different types of vacuoles may perform these different functions.

The existence of different vacuoles also opens complementary questions about vacuole generation and control of the vacuolar content. The studies devoted to finding an answer to this question are complicated by the fact that isolation and evacuolation of cells (protoplast isolation and culture) induces stress that results in changes in the nature of the vacuolar environment and content.

Mutants in which the process of vacuolar genesis and/or the control of the internal vacuolar environment are affected are highly valuable to allow the study of these phenomena in intact cells in the original tissue. Mutants of this type are not well described in the literature. This has hampered research in this area.

Flower color is predominantly due to three types of pigment: flavonoids, carotenoids and betalains. Of the three, the flavonoids are the most common and contribute a range of colors from yellow to red to blue. The flavonoid pigments are secondary metabolites of the phenylpropanoid pathway. The biosynthetic pathway for the flavonoid pigments (flavonoid pathway) is well established, (Holton and Cornish, Plant Cell 7:1071-1083, 1995; Mol et al, Trends Plant Sci. 3: 212-217, 1998; Winkel-Shirley, Plant Physiol. 126:485-493, 2001a; Winkel-Shirley, Plant Physiol. 127:1399-1404, 2001b, Tanaka et al, Plant Cell, Tissue and Organ Culture 80 (1):1-24, 2005, Koes et al. Trends in Plant Science, May 2005).

The flavonoid molecules that make the major contribution to flower or fruit color are the anthocyanins, which are glycosylated derivatives of anthocyanidins. Anthocyanins are generally localized in the vacuole of the epidermal cells of petals or fruits or the vacuole of the sub epidermal cells of leaves. Anthocyanins can be further modified through the addition of glycosyl groups, acyl groups and methyl groups. The final visible color of a flower or fruit is generally a combination of a number of factors including the type of anthocyanin accumulating, modifications to the anthocyanidin molecule, co-pigmentation with other flavonoids such as flavonols and flavones, complexation with metal ions and the pH of the vacuole.

The vacuolar pH is a factor in anthocyanin stability and color. Although a neutral to alkaline pH generally yields bluer anthocyanidin colors, these molecules are less stable at this pH.

Vacuoles, occupy a large part of the plant cell volume and play a crucial role in the maintenance of cell homeostasis. In mature cells, these organelles can approach 90% of the total cell volume, can store a large variety of molecules (ions, organic acids, sugar, enzymes, storage proteins and different types of secondary metabolites) and serve as reservoirs of protons and other metabolically important ions. Different transporters on the membrane of the vacuoles regulate the accumulation of solutes in this compartment and drive the accumulation of water producing the turgor of the cell. These structurally simple organelles play a wide range of essential roles in the life of a plant and this requires their internal environment to be tightly regulated.

There is a need to be able to manipulate vacuolar pH in plant cells and organelles in order to generate desired flower colors.

### SUMMARY OF THE INVENTION

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

Nucleotide and amino acid sequences are referred to by a sequence identifier number (SEQ ID NO:). The SEQ ID NOs: correspond numerically to the sequence identifiers <400>1 (SEQ ID NO:1), <400>2 (SEQ ID NO:2), etc. A summary of the sequence identifiers is provided in Table 1. A sequence listing is provided after the claims.

The present invention provides a novel nucleic acid molecule encoding a polypeptide having pH modulating or altering activity and to the use of the nucleic acid molecules and/or corresponding polypeptides to generate genetic agents or constructs or other molecules which manipulate vacuolar pH in a cell, groups of cells, organelles, parts or reproductions of a plant. Manipulation of the pH pathway, and optionally, together with manipulation of the anthocyanin pathway provides a powerful technique to generate novel colors or traits, especially in carnation and rose.

Accordingly, the present invention provides genetic agents and proteinaceous agents which increase or decrease the level of acidity or alkalinity in the vacuole of a plant cell. The ability to alter pH of the vacuole enables manipulation of flower color. The agents include nucleic acid molecules such as cDNA and genomic DNA or parts or fragments thereof, antisense, sense or RNAi molecules or complexes comprising same, ribozymes, peptides and proteins.

The present invention further provides a nucleic acid comprising a sequence of nucleotides encoding or complementary to a sequence encoding a protein which exhibits a direct or indirect effect on vacuolar pH.

The present invention enables, therefore, levels of expression of the subject nucleic acid molecules to be manipulated or to introduce the nucleic acid to a plant cell in order to alter vacuolar pH. This in turn permits flower color or taste or other characteristics to be manipulated.

The present invention provides, therefore, genetically modified plants exhibiting altered flower color or taste or other characteristics. Reference to genetically modified plants exhibiting altered flower color or taste or other characteristics. Reference to genetically modified plants includes the first generation plant or plantlet as well as progeny and subsequent generations of the plant. Reference to a “plant” includes reference to plant parts including reproductive portions, seeds, flowers, stems, leaves, stalks, pollen and germ plasm, callus including immature and mature callus.

A particularly preferred aspect of the present invention relates to down regulation of the pH modulating or altering genetic and proteinaceous agents capable of modulating or altering the level of acidity or alkalinity, leading to an increase in vacuolar pH in a plant, resulting in bluer colored flowers in said plant.

The present invention further contemplates cut flowers including severed stems containing flowers of the genetically altered plants or their progeny in isolated form or packaged for sale or arranged on display.

A summary of sequence identifiers used throughout the subject specification is provided in Table 1:

TABLE 1
Summary of sequence identifiers

| SEQ | | | |
|---|---|---|---|
| ID NO: | Sequence name | Type of sequence | Description |
| 1 | MAC F55.nt | nucleotide | PPM1 cDNA clone |
| 2 | MAC F55.aa | amino acid | translation of |
| | | | PPM1 cDNA |
| 3 | MAC 9F1.nt | nucleotide | |
| 4 | MAC 9F1.aa | amino acid | |
| 5 | CAC16.5.nt | nucleotide | |
| 6 | CAC 16.5.aa | amino acid | |
| 7 | Mse A1 | nucleotide | primer |
| 8 | MseA2 | nucleotide | primer |
| 9 | mse + 0 | nucleotide | primer |
| 10 | Mse + A | nucleotide | primer |
| 11 | Mse + C | nucleotide | primer |
| 12 | Mse + G | nucleotide | primer |
| 13 | Mse + T | nucleotide | primer |
| 14 | Eco + A1 | nucleotide | primer |
| 15 | Eco + A2 | nucleotide | primer |
| 16 | Eco + A | nucleotide | primer |
| 17 | Eco + C | nucleotide | primer |
| 18 | Eco + G | nucleotide | primer |
| 19 | Eco + T | nucleotide | primer |
| 20 | Mse + AA | nucleotide | primer |
| 21 | Mse + AC | nucleotide | primer |
| 22 | Mse + AG | nucleotide | primer |
| 23 | Mse + AT | nucleotide | primer |
| 24 | Mse + CA | nucleotide | primer |
| 25 | Mse + CC | nucleotide | primer |
| 26 | Mse + CG | nucleotide | primer |
| 27 | Mse + CT | nucleotide | primer |
| 28 | Mse + GA | nucleotide | primer |
| 29 | Mse + GC | nucleotide | primer |
| 30 | Mse + GG | nucleotide | primer |
| 31 | Mse + GT | nucleotide | primer |
| 32 | Mse + TA | nucleotide | primer |
| 33 | Mse + TC | nucleotide | primer |
| 34 | Mse + TG | nucleotide | primer |
| 35 | Mse + TT | nucleotide | primer |
| 36 | Eco + AA | nucleotide | primer |
| 37 | Eco + AC | nucleotide | primer |
| 38 | Eco + AG | nucleotide | primer |
| 39 | Eco + AT | nucleotide | primer |
| 40 | Eco + CA | nucleotide | primer |
| 41 | Eco + CC | nucleotide | primer |
| 42 | Eco + CG | nucleotide | primer |
| 43 | Eco + CT | nucleotide | primer |
| 44 | Eco + GA | nucleotide | primer |
| 45 | Eco + GC | nucleotide | primer |
| 46 | Eco + GG | nucleotide | primer |
| 47 | Eco + GT | nucleotide | primer |
| 48 | Eco + TA | nucleotide | primer |
| 49 | Eco + TC | nucleotide | primer |
| 50 | Eco + TG | nucleotide | primer |
| 51 | Eco + TT | nucleotide | primer |
| 52 | 1702 | nucleotide | primer |
| 53 | 1703 | nucleotide | primer |
| 54 | 1741 | nucleotide | primer |
| 55 | 1742 | nucleotide | primer |
| 56 | 1750 | nucleotide | primer |
| 57 | 1788 | nucleotide | primer |
| 58 | 1789 | nucleotide | primer |
| 59 | 1812 | nucleotide | primer |
| 60 | 1831 | nucleotide | primer |
| 61 | 1832 | nucleotide | primer |
| 62 | 1847 | nucleotide | primer |
| 63 | 1848 | nucleotide | primer |
| 64 | 1861 | nucleotide | primer |
| 65 | 1864 | nucleotide | primer |
| 66 | 1885 | nucleotide | primer |
| 67 | 1886 | nucleotide | primer |
| 68 | 1956 | nucleotide | primer |
| 69 | 2035 | nucleotide | primer |
| 70 | 2037 | nucleotide | primer |
| 71 | 2038 | nucleotide | primer |
| 72 | 2039 | nucleotide | primer |
| 73 | 2040 | nucleotide | primer |
| 74 | 2073 | nucleotide | primer |
| 75 | 2075 | nucleotide | primer |
| 76 | 2078 | nucleotide | primer |
| 77 | 2123 | nucleotide | primer |
| 78 | 2124 | nucleotide | primer |
| 79 | 2196 | nucleotide | primer |
| 80 | 2270 | nucleotide | primer |
| 81 | 2271 | nucleotide | primer |
| 82 | 1706 | nucleotide | primer |
| 83 | 1707 | nucleotide | primer |
| 84 | 1743 | nucleotide | primer |
| 85 | 1768 | nucleotide | primer |
| 86 | 1876 | nucleotide | primer |
| 87 | 1877 | nucleotide | primer |
| 88 | 1878 | nucleotide | primer |
| 89 | 2061 | nucleotide | primer |
| 90 | 2101 | nucleotide | primer |
| 91 | 2178 | nucleotide | primer |
| 92 | 1654 | nucleotide | primer |
| 93 | 1655 | nucleotide | primer |
| 94 | 1769 | nucleotide | primer |
| 95 | 1770 | nucleotide | primer |
| 96 | 1870 | nucleotide | primer |
| 97 | 1871 | nucleotide | Primer |
| 98 | 1-2contig.fa | nucleotide | Rose PPM1 |
| | | | homologue cDNA |
| 99 | 1-2protein.fa | amino acid | Translation of |
| | | | Rose PPM1 |
| | | | homologue cDNA |
| 100 | #2124: 5′ | nucleotide | primer |
| 101 | #2078: 5′ | nucleotide | primer |

### BRIEF DESCRIPTION OF THE FIGURES

FIG. 1 is a diagrammatical representation of replicon pK7GWIWG2(I) PPM1-1 10639 bp.

FIG. 2 is a diagrammatical representation of replicon pK7GWIWG2(I) PPM1-2 1171 bp.

FIG. 3 is a diagrammatical representation of replicon pK7GWIWG2(I) MAC9F1 10801 bp.

FIG. 4 is a diagrammatical representation of replicon pK7GWIWG2(I) CAC16.5 10763 bp.

FIG. 5 is a photographic representation of an autoradiograph of a Southern blot probed with ³²P-labelled Rose PPM1 fragment. Each lane contained 10 μg of DNA digested with EcoRI. Washing conditions were: twice in 6×SSC/1% SDS at 50° C. for 1 hour. Lanes contain DNA from: M: markers, 1: Anemone, 2: Carnation, 3: Chrysanthemum, 4: Gerbera, 5: Hyacinth, 6: Iris, 7: Liatrus, 8: Pansy (Viola), 9: Petunia, 10: Nierembergia, 11: Rose, 12: Tobacco

FIG. 6 is a photographic representation of an autoradiograph of a Southern blot probed with ³²P-labelled Petunia CAC16.5 fragment. Each lane contained 10 μg of DNA digested with EcoRI. Washing conditions were: 6×SSC/1% SDS at 50° C. for 30 mins. Lanes contain DNA from: M: markers, 1: Anemone, 2: Carnation, 3: Chrysanthemum, 4: Gerbera, 5: Hyacinth, 6: Iris, 7: Liatrus, 8: Pansy (Viola), 9: Petunia, 10: Nierembergia, 11: Rose, 12: Tobacco

FIG. 7 is a photographic representation of an autoradiograph of a Southern blot probed with ³²P-labelled Petunia MAC9F1 fragment. Each lane contained 10 μg of DNA digested with EcoRI. Washing conditions were: 6×SSC/1% SDS at 50° C. for 30 mins. Lanes contain DNA from: M: markers, 1: Anemone, 2: Carnation, 3: Chrysanthemum, 4: Gerbera, 5: Hyacinth, 6: Iris, 7: Liatrus, 8: Pansy (Viola), 9: Petunia, 10: Nierembergia, 11: Rose, 12: Tobacco

FIG. 8 is a diagrammatical representation of replicon pBinPLUS.

FIG. 9 is a diagrammatical representation of replicon pBluescript.

FIG. 10 is a diagrammatical representation of replicon pCGP1275.

FIG. 11 is a diagrammatical representation of replicon pWTT2132 p.

FIG. 12 is a diagrammatical representation of replicon pWTT2132 19.5 kb Xhol (blunt).

FIG. 13 is a diagrammatical representation of replicon pBinPLUS 12.3 kb KpnI (blunt).

FIG. 14 is a diagrammatical representation of replicon pWTT2132.

FIG. 15 is a diagrammatical representation of replicon pCGP2355 26.8 kb HincII (blunt).

FIG. 16 is a diagrammatical representation of replicon pRTppoptcAFP EcoRI/XbaI 3.3 kb.

FIG. 17 is a diagrammatical representation of replicon pCGP2756 3.3 kb.

FIG. 18 is a diagrammatical representation of replicon pWTT2132 19.5 kb PstI.

FIG. 19 is a diagrammatical representation of replicon pBinPLUS 12.3 kb HindIII.

FIG. 20 is a diagrammatical representation of replicon pCGP2355 26.8 kb HincII (blunt).

### DETAILED DESCRIPTION OF THE INVENTION

In accordance with the present invention, nucleic acid sequences encoding polypeptides having pH modulating or altering activities have been identified, cloned and assessed. The recombinant genetic sequences of the present invention permit the modulation of expression of genes or nucleic acids encoding pH modulating or altering activities by, for example, de novo expression, over-expression, sense suppression, antisense inhibition, ribozyme, minizyme and DNAzyme activity, RNAi-induction or methylation-induction or other transcriptional or post-transcriptional silencing activities. RNAi-induction includes genetic molecules such as hairpin, short double stranded DNA or RNA, and partially double stranded DNAs or RNAs with one or two single stranded nucleotide over hangs. The ability to control vacuolar pH in plants thereby enables the manipulation of petal color in response to pH change. Moreover, the present invention extends to plants and reproductive or vegetative parts thereof including flowers, fruits, seeds, vegetables, leaves, stems and the like. The present invention further extends to ornamental transgenic or genetically modified plants. The term “transgenic” also includes progeny plants and plants from subsequent genetic manipulation and/or crosses thereof from the primary transgenic plants.

Accordingly, one aspect of the present invention provides an isolated nucleic acid molecule comprising a sequence of nucleotides encoding or complementary to a sequence encoding a pH modulating or altering gene or a polypeptide having pH modulating or altering activity wherein expression of said nucleic acid molecule alters or modulates pH inside the cell or vacuole.

Preferably the nucleic acid of the present invention modulates vacuolar pH.

Another aspect of the present invention contemplates an isolated nucleic acid molecule comprising a sequence of nucleotides encoding or complementary to a sequence encoding a pH modulating or altering gene operably linked to a nucleic acid sequence comprising a sequence of nucleotides encoding or complementary to a sequence encoding an anthocyanin pathway gene.

By the term “nucleic acid molecule” is meant a genetic sequence in a non-naturally occurring condition. Generally, this means isolated away from its natural state or synthesized or derived in a non-naturally-occurring environment. More specifically, it includes nucleic acid molecules formed or maintained in vitro, including genomic DNA fragments recombinant or synthetic molecules and nucleic acids in combination with heterologous nucleic acids. It also extends to the genomic DNA or cDNA or part thereof encoding F3′5′H or a part thereof in reverse orientation relative to its own or another promoter. It further extends to naturally occurring sequences following at least a partial purification relative to other nucleic acid sequences.

The term “genetic sequences” is used herein in its most general sense and encompasses any contiguous series of nucleotide bases specifying directly, or via a complementary series of bases, a sequence of amino acids in a pH modulating protein. Such a sequence of amino acids may constitute a full-length pH modulating or altering enzyme such as is set forth in SEQ ID NO: 2, 4 or 6 or an amino acid sequence having at least 50% similarity thereto such as SEQ ID NO: 99, or an active truncated form thereof or may correspond to a particular region such as an N-terminal, C-terminal or internal portion of the enzyme. A genetic sequence may also be referred to as a sequence of nucleotides or a nucleotide sequence and includes a recombinant fusion of two or more sequences.

In accordance with the above aspects of the present invention there is provided a nucleic acid molecule comprising a nucleotide sequence or complementary nucleotide sequence substantially as set forth in SEQ ID NO: 1, 3 or 5 or having at least about 50% similarity thereto or capable of hybridizing to the sequence set forth in SEQ ID NO: 1, 3 or 5 under low stringency conditions such as SEQ ID NO: 98.

In accordance with the present invention, the anthocyanin pathway genes optionally contemplated to be used in conjunction with the pH modulating or altering nucleic acids, set forth in SEQ ID NO: 1, 3 or 5 or having at least about 50% similarity thereto or capable of hybridizing to the sequence set forth in SEQ ID NO: 1, 3 or 5 under low stringency conditions, have been previously described in a series of patents and application from the same assignee (including, for example, patents and patent application for the families relating to PCT/AU92/00334; PCTAU96/00296; PCT/AU93/00127; PCT/AU97/00124; PCT/AU93/00387; PCT/AU93/00400; PCT/AU01/00358; PCT/AU03/00079; PCT/AU03/01111; JP 2003-293121)

Table 1 provides a summary of the sequence identifiers.

Alternative percentage similarities and identities (at the nucleotide or amino acid level) encompassed by the present invention include at least about 60% or at least about 65% or at least about 70% or at least about 75% or at least about 80% or at least about 85% or at least about 90% or above, such as about 95% or about 96% or about 97% or about 98% or about 99%, such as at least about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%.

In a particularly preferred embodiment, there is provided an isolated nucleic acid molecule comprising a nucleotide sequence or complementary nucleotide sequence substantially as set forth in SEQ ID NO: 1, 3 or 5 or having at least about 50% similarity thereto or capable of hybridizing to the sequence set forth in SEQ ID NO: 1, 3 or 5 or complementary strands of either under low stringency conditions, wherein said nucleotide sequence encodes a polypeptide having pH modulating or altering activity.

For the purposes of determining the level of stringency to define nucleic acid molecules capable of hybridizing to SEQ ID NO: 1, 3 or 5 reference herein to a low stringency includes and encompasses from at least about 0% to at least about 15% v/v formamide and from at least about 1M to at least about 2 M salt for hybridization, and at least about 1 M to at least about 2 M salt for washing conditions. Generally, low stringency is from about 25-30° C. to about 42° C. The temperature may be altered and higher temperatures used to replace the inclusion of formamide and/or to give alternative stringency conditions. Alternative stringency conditions may be applied where necessary, such as medium stringency, which includes and encompasses from at least about 16% v/v to at least about 30% v/v formamide and from at least about 0.5 M to at least about 0.9 M salt for hybridization, and at least about 0.5 M to at least about 0.9 M salt for washing conditions, or high stringency, which includes and encompasses from at least about 31% v/v to at least about 50% v/v formamide and from at least about 0.01 M to at least about 0.15 M salt for hybridization, and at least about 0.01 M to at least about 0.15 M salt for washing conditions. In general, washing is carried out Tₘ=69.3+0.41 (G+C) % (Marmur and Doty, J. Mol. Biol. 5: 109, 1962). However, the Tₘ of a duplex DNA decreases by 1° C. with every increase of 1% in the number of mismatch base pairs (Bonner and Laskey, Eur. J. Biochem. 46: 83, 1974). Formamide is optional in these hybridization conditions. Accordingly, particularly preferred levels of stringency are defined as follows: low stringency is 6×SSC buffer, 1.0% w/v SDS at 25-42° C.; a moderate stringency is 2×SSC buffer, 1.0% w/v SDS at a temperature in the range 20° C. to 65° C.; high stringency is 0.1×SSC buffer, 0.1% w/v SDS at a temperature of at least 65° C.

Another aspect of the present invention provides a nucleic acid molecule comprising a sequence of nucleotides encoding or complementary to a sequence encoding an amino acid sequence substantially as set forth in SEQ ID NO: 2, 4 or 6 or an amino acid sequence having at least about 50% similarity thereto.

The term similarity as used herein includes exact identity between compared sequences at the nucleotide or amino acid level. Where there is non-identity at the nucleotide level, similarity includes differences between sequences which result in different amino acids that are nevertheless related to each other at the structural, functional, biochemical and/or conformational levels. Where there is non-identity at the amino acid level, similarity includes amino acids that are nevertheless related to each other at the structural, functional, biochemical and/or conformational levels. In a particularly preferred embodiment, nucleotide and sequence comparisons are made at the level of identity rather than similarity.

Terms used to describe sequence relationships between two or more polynucleotides or polypeptides include “reference sequence”, “comparison window”, “sequence similarity”, “sequence identity”, “percentage of sequence similarity”, “percentage of sequence identity”, “substantially similar” and “substantial identity”. A “reference sequence” is at least 12 but frequently 15 to 18 and often at least 25 or above, such as 30 monomer units, inclusive of nucleotides and amino acid residues, in length. Because two polynucleotides may each comprise (1) a sequence (i.e. only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of typically 12 contiguous residues that is compared to a reference sequence. The comparison window may comprise additions or deletions (i.e. gaps) of about 20% or less as compared to the reference sequence (which does not comprise additions or deletions) for optimal alignment of the two sequences. Optimal alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e. resulting in the highest percentage homology over the comparison window) generated by any of the various methods selected. Reference also may be made to the BLAST family of programs as, for example, disclosed by Altschul et al. (Nucl. Acids Res. 25: 3389-3402, 1997). A detailed discussion of sequence analysis can be found in Unit 19.3 of Ausubel et al. (“Current Protocols in Molecular Biology” John Wiley & Sons Inc, 1994-1998, Chapter 15, 1998).

The terms “sequence similarity” and “sequence identity” as used herein refers to the extent that sequences are identical or functionally or structurally similar on a nucleotide-by-nucleotide basis or an amino acid-by-amino acid basis over a window of comparison. Thus, a “percentage of sequence identity”, for example, is calculated by comparing two optimally aligned sequences over the window of comparison, determining the number of positions at which the identical nucleic acid base (e.g. A, T, C, G, I) or the identical amino acid residue (e.g. Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. For the purposes of the present invention, “sequence identity” will be understood to mean the “match percentage” calculated by the DNASIS computer program (Version 2.5 for windows; available from Hitachi Software engineering Co., Ltd., South San Francisco, Calif., USA) using standard defaults as used in the reference manual accompanying the software. Similar comments apply in relation to sequence similarity.

The nucleic acid sequences contemplated herein also encompass oligonucleotides useful as genetic probes for amplification reactions or as antisense or sense molecules capable of regulating expression of the corresponding gene in a plant. Sense molecules include hairpin constructs, short double stranded DNAs and RNAs and partially double stranded DNAs and RNAs which one or more single stranded nucleotide over hangs. An antisense molecule as used herein may also encompass a genetic construct comprising the structural genomic or cDNA gene or part thereof in reverse orientation relative to its own or another promoter. It may also encompass a homologous genetic sequence. An antisense or sense molecule may also be directed to terminal or internal portions of the gene encoding a polypeptide having a pH modulating or altering activity or to combinations of the above such that the expression of the gene is reduced or eliminated.

With respect to this aspect of the invention, there is provided an oligonucleotide of 5-50 nucleotides such as 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55 having substantial similarity to a part or region of a molecule with a nucleotide sequence set forth in SEQ ID NO: 1, 3 or 5. By substantial similarity or complementarity in this context is meant a hybridizable similarity under low, alternatively and preferably medium and alternatively and most preferably high stringency conditions specific for oligonucleotide hybridization (Sambrook et al, Molecular Cloning: A Laboratory Manual, 2^{nd} edition, Cold Spring Harbor Laboratories, Cold Spring Harbor, N.Y., USA, 1989). Such an oligonucleotide is useful, for example, in screening for pH modulating or altering genetic sequences from various sources or for monitoring an introduced genetic sequence in a transgenic plant. The preferred oligonucleotide is directed to a conserved pH modulating or altering genetic sequence or a sequence conserved within a plant genus, plant species and/or plant variety.

In one aspect of the present invention, the oligonucleotide corresponds to the 5′ or the 3′ end of the nucleic acid modulating or altering pH sequences. For convenience, the 5′ end is considered herein to define a region substantially between the start codon of the structural gene to a centre portion of the gene, and the 3′ end is considered herein to define a region substantially between the centre portion of the gene and the terminating codon of the structural gene. It is clear, therefore, that oligonucleotides or probes may hybridize to the 5′ end or the 3′ end or to a region common to both the 5′ and the 3′ ends. The present invention extends to all such probes.

In one embodiment, the nucleic acid sequence encoding a pH modulating or altering proteins or various functional derivatives thereof is used to reduce the level of an endogenous pH modulating or altering protein (e.g. via co-suppression or antisense-mediated suppression) or other post-transcriptional gene silencing (PTGS) processes including RNAi or alternatively the nucleic acid sequence encoding this enzyme or various derivatives or parts thereof is used in the sense or antisense orientation to reduce the level of a pH modulating or altering protein. The use of sense strands, double or partially single stranded such as constructs with hairpin loops is particularly useful in inducing a PTGS response. In a further alternative, ribozymes, minizymes or DNAzymes could be used to inactivate target nucleic acid sequences.

Still a further embodiment encompasses post-transcriptional inhibition to reduce translation into polypeptide material. Still yet another embodiment involves specifically inducing or removing methylation.

Reference herein to the changing of a pH modulating or altering activity relates to an elevation or reduction in activity of up to 30% or more preferably of 30-50%, or even more preferably 50-75% or still more preferably 75% or greater above or below the normal endogenous or existing levels of activity. Such elevation or reduction may be referred to as modulation or alteration of a pH modulating protein. Often, modulation is at the level of transcription or translation of pH modulating or altering genetic sequences.

The nucleic acids of the present invention may be a ribonucleic acid or deoxyribonucleic acids, single or double stranded and linear or covalently closed circular molecules. Preferably, the nucleic acid molecule is cDNA. The present invention also extends to other nucleic acid molecules which hybridize under low, preferably under medium and most preferably under high stringency conditions with the nucleic acid molecules of the present invention and in particular to the sequence of nucleotides set forth in SEQ ID NO: 1, 3 or 5 or a part or region thereof. In its most preferred embodiment, the present invention extends to a nucleic acid molecule having a nucleotide sequence set forth in SEQ ID NO: 1, 3 or 5 or to a molecule having at least 40%, more preferably at least 45%, even more preferably at least 55%, still more preferably at least 65%-70%, and yet even more preferably greater than 85% similarity at the level of nucleotide or amino acid sequence to at least one or more regions of the sequence set forth in SEQ ID NO: 1, 3 or 5 and wherein the nucleic acid encodes or is complementary to a sequence which encodes an enzyme having a pH modulating or altering activity. It should be noted, however, that nucleotide or amino acid sequences may have similarities below the above given percentages and yet still encode a pH modulating or altering activity and such molecules may still be considered in the scope of the present invention where they have regions of sequence conservation. The present invention further extends to nucleic acid molecules in the form of oligonucleotide primers or probes capable of hybridizing to a portion of the nucleic acid molecules contemplated above, and in particular those set forth in SEQ ID NO: 1, 3 or 5, under low, preferably under medium and most preferably under high stringency conditions. Preferably the portion corresponds to the 5′ or the 3′ end of the gene. For convenience the 5′ end is considered herein to define a region substantially between the start codon of the structural genetic sequence to a centre portion of the gene, and the 3′ end is considered herein to define a region substantially between the centre portion of the gene and the terminating codon of the structural genetic sequence. It is clear, therefore, that oligonucleotides or probes may hybridize to the 5′ end or the 3′ end or to a region common to both the 5′ and the 3′ ends. The present invention extends to all such probes.

The term gene is used in its broadest sense and includes cDNA corresponding to the exons of a gene. Accordingly, reference herein to a gene is to be taken to include:—

(i) a classical genomic gene consisting of transcriptional and/or translational regulatory sequences and/or a coding region and/or non-translated sequences (i.e. introns, 5′- and 3′-untranslated sequences); or
(ii) mRNA or cDNA corresponding to the coding regions (i.e. exons) and 5′- and 3′-untranslated sequences of the gene.

The term gene is also used to describe synthetic or fusion molecules encoding all or part of an expression product. In particular embodiments, the term nucleic acid molecule and gene may be used interchangeably.

The nucleic acid or its complementary form may encode the full-length enzyme or a part or derivative thereof. By “derivative” is meant any single or multiple amino acid substitutions, deletions, and/or additions relative to the naturally occurring enzyme and which retains a pH modulating or altering activity. In this regard, the nucleic acid includes the naturally occurring nucleotide sequence encoding a pH modulating or altering activity or may contain single or multiple nucleotide substitutions, deletions and/or additions to said naturally occurring sequence. The nucleic acid of the present invention or its complementary form may also encode a “part” of the pH modulating or altering protein, whether active or inactive, and such a nucleic acid molecule may be useful as an oligonucleotide probe, primer for polymerase chain reactions or in various mutagenic techniques, or for the generation of antisense molecules.

Reference herein to a “part” of a nucleic acid molecule, nucleotide sequence or amino acid sequence, preferably relates to a molecule which contains at least about 10 contiguous nucleotides or five contiguous amino acids, as appropriate.

Amino acid insertional derivatives of the pH modulating or altering protein of the present invention include amino and/or carboxyl terminal fusions as well as intra-sequence insertions of single or multiple amino acids. Insertional amino acid sequence variants are those in which one or more amino acid residues are introduced into a predetermined site in the protein although random insertion is also possible with suitable screening of the resulting product. Deletional variants are characterized by the removal of one or more amino acids from the sequence. Substitutional amino acid variants are those in which at least one residue in the sequence has been removed and a different residue inserted in its place. Typical substitutions are those made in accordance with Table 2.

TABLE 2
Suitable residues for amino acid substitutions

| Original residue | Exemplary substitutions |
|---|---|
| Ala | Ser |
| Arg | Lys |
| Asn | Gln; His |
| Asp | Glu |
| Cys | Ser |
| Gln | Asn; Glu |
| Glu | Asp |
| Gly | Pro |
| His | Asn; Gln |
| Ile | Leu; Val |
| Leu | Ile; Val |
| Lys | Arg; Gln; Glu |
| Met | Leu; Ile; Val |
| Phe | Met; Leu; Tyr |
| Ser | Thr |
| Thr | Ser |
| Trp | Tyr |
| Tyr | Trp; Phe |
| Val | Ile; Leu; Met |

Where the pH modulating or altering protein is derivatized by amino acid substitution, the amino acids are generally replaced by other amino acids having like properties, such as hydrophobicity, hydrophilicity, electronegativity, bulky side chains and the like. Amino acid substitutions are typically of single residues. Amino acid insertions will usually be in the order of about 1-10 amino acid residues and deletions will range from about 1-20 residues. Preferably, deletions or insertions are made in adjacent pairs, i.e. a deletion of two residues or insertion of two residues.

The amino acid variants referred to above may readily be made using peptide synthetic techniques well known in the art, such as solid phase peptide synthesis (Merrifield, J. Am. Chem. Soc. 85:2149, 1964) and the like, or by recombinant DNA manipulations.

Techniques for making substitution mutations at predetermined sites in DNA having known or partially known sequence are well known and include, for example, M13 mutagenesis. The manipulation of DNA sequence to produce variant proteins which manifest as substitutional, insertional or deletional variants are conveniently described, for example, in Sambrook et al. (1989, supra).

Other examples of recombinant or synthetic mutants and derivatives of the pH modulating or altering proteins of the present invention include single or multiple substitutions, deletions and/or additions of any molecule associated with the enzyme such as carbohydrates, lipids and/or proteins or polypeptides.

The terms “analogs” and “derivatives” also extend to any functional chemical equivalent of pH modulating or altering proteins and also to any amino acid derivative described above. For convenience, reference to pH modulating or altering proteins herein includes reference to any functional mutant, derivative, part, fragment, homolog or analog thereof. The present invention is exemplified using nucleic acid sequences derived from pansy, salvia, sollya or lavender or kennedia since this represents the most convenient and preferred source of material to date. However, one skilled in the art will immediately appreciate that similar sequences can be isolated from any number of sources such as other plants or certain microorganisms. All such nucleic acid sequences encoding directly or indirectly a pH modulating protein are encompassed by the present invention regardless of their source. Examples of other suitable sources of genes encoding pH modulating or altering proteins include, but are not limited to Dianthus spp. Rosa spp. Chrysanthemum spp. Cyclamen spp., Gerbera spp., Iris spp., Pelargonium spp., Liparieae, Geranium spp., Saintpaulia spp., Plumbago spp., etc.

In accordance with the present invention, a nucleic acid sequence encoding a pH modulating or altering protein may be introduced into and expressed in a transgenic plant in either orientation thereby providing a means to modulate or alter the vacuolar pH by either reducing or eliminating endogenous or existing pH modulating or altering protein activity thereby allowing the vacuolar pH to increase. A particularly preferred effect is a visible effect of a shift to blue in the color of the anthocyanins and/or in the resultant flower color. Expression of the nucleic acid sequence in the plant may be constitutive, inducible or developmental and may also be tissue-specific. The word “expression” is used in its broadest sense to include production of RNA or of both RNA and protein. It also extends to partial expression of a nucleic acid molecule.

According to this aspect of the present invention, there is provided a method for producing a transgenic flowering plant capable of synthesizing a pH modulating or altering protein, said method comprising stably transforming a cell of a suitable plant with a nucleic acid sequence which comprises a sequence of nucleotides encoding said pH modulating or altering proteins under conditions permitting the eventual expression of said nucleic acid sequence, regenerating a transgenic plant from the cell and growing said transgenic plant for a time and under conditions sufficient to permit the expression of the nucleic acid sequence. The transgenic plant may thereby produce non-indigenous pH modulating or altering proteins at elevated levels relative to the amount expressed in a comparable non-transgenic plant.

Another aspect of the present invention contemplates a method for producing a transgenic plant with reduced indigenous or existing pH modulating or altering activity, said method comprising stably transforming a cell of a suitable plant with a nucleic acid molecule which comprises a sequence of nucleotides encoding or complementary to a sequence encoding a pH modulating activity, regenerating a transgenic plant from the cell and where necessary growing said transgenic plant under conditions sufficient to permit the expression of the nucleic acid.

Yet another aspect of the present invention contemplates a method for producing a genetically modified plant with reduced indigenous or existing pH modulating or altering protein activity, said method comprising altering the pH modulating or altering gene through modification of the indigenous sequences via homologous recombination from an appropriately altered pH modulating or altering gene or derivative or part thereof introduced into the plant cell, and regenerating the genetically modified plant from the cell.

As used herein an “indigenous” enzyme is one, which is native to or naturally expressed in a particular cell. A “non-indigenous” enzyme is an enzyme not native to the cell but expressed through the introduction of genetic material into a plant cell, for example, through a transgene. An “endogenous” enzyme is an enzyme produced by a cell but which may or may not be indigenous to that cell.

In a preferred embodiment, the present invention contemplates a method for producing a transgenic flowering plant exhibiting altered floral or inflorescence properties, said method comprising stably transforming a cell of a suitable plant with a nucleic acid sequence of the present invention, regenerating a transgenic plant from the cell and growing said transgenic plant for a time and under conditions sufficient to permit the expression of the nucleic acid sequence.

The term “inflorescence” as used herein refers to the flowering part of a plant or any flowering system of more than one flower which is usually separated from the vegetative parts by an extended internode, and normally comprises individual flowers, bracts and peduncles, and pedicels. As indicated above, reference to a “transgenic plant” may also be read as a “genetically modified plant”.

Alternatively, said method may comprise stably transforming a cell of a suitable plant with a nucleic acid sequence of the present invention or its complementary sequence, regenerating a transgenic plant from the cell and growing said transgenic plant for a time and under conditions sufficient to alter the level of activity of the indigenous or existing pH modulating or altering proteins. Preferably the altered level would be less than the indigenous or existing level of pH modulating or altering activity in a comparable non-transgenic plant. Without wishing to limit the present invention, one theory of mode of action is that reduction of the indigenous pH modulating protein activity requires the expression of the introduced nucleic acid sequence or its complementary sequence. However, expression of the introduced genetic sequence or its complement may not be required to achieve the desired effect: namely, a flowering plant exhibiting altered floral or inflorescence properties.

In a related embodiment, the present invention contemplates a method for producing a flowering plant exhibiting altered floral or inflorescence properties, said method comprising alteration of the pH modulating or altering gene through modification of the indigenous sequences via homologous recombination from an appropriately altered pH modulating or altering gene or derivative or part thereof introduced into the plant cell, and regenerating the genetically modified plant from the cell.

Preferably, the altered floral or inflorescence includes the production of different shades of blue or purple or red flowers or other colors, depending on the genotype and physiological conditions of the recipient plant.

Accordingly, the present invention extends to a method for producing a transgenic plant capable of expressing a recombinant gene encoding a pH modulating or altering protein or part thereof or which carries a nucleic acid sequence which is substantially complementary to all or a part of a mRNA molecule encoding a pH modulating or altering protein, said method comprising stably transforming a cell of a suitable plant with the isolated nucleic acid molecule comprising a sequence of nucleotides encoding, or complementary to a sequence encoding, a pH modulating or altering protein, where necessary under conditions permitting the eventual expression of said isolated nucleic acid molecule, and regenerating a transgenic plant from the cell.

One skilled in the art will immediately recognise the variations applicable to the methods of the present invention, such as increasing or decreasing the expression of the enzyme naturally present in a target plant leading to differing shades of colors such as different shades of blue, purple or red.

The present invention, therefore, extends to all transgenic plants or parts or cells therefrom of transgenic plants or progeny of the transgenic plants containing all or part of the nucleic acid sequences of the present invention, or antisense forms thereof and/or any homologs or related forms thereof and, in particular, those transgenic plants which exhibit altered floral or inflorescence properties. The transgenic plants may contain an introduced nucleic acid molecule comprising a nucleotide sequence encoding or complementary to a sequence encoding a pH modulating or altering protein. Generally, the nucleic acid would be stably introduced into the plant genome, although the present invention also extends to the introduction of a pH modulating or altering nucleotide sequence within an autonomously-replicating nucleic acid sequence such as a DNA or RNA virus capable of replicating within the plant cell. The invention also extends to seeds from such transgenic plants. Such seeds, especially if colored, are useful as proprietary tags for plants. Any and all methods for introducing genetic material into plant cells including but not limited to Agrobacterium-mediated transformation, biolistic particle bombardment etc. are encompassed by the present invention.

Another aspect of the present invention contemplates the use of the extracts from transgenic plants or plant parts or cells therefrom of transgenic plants or progeny of the transgenic plants containing all or part of the nucleic acid sequences of the present invention and, in particular, the extracts from those transgenic plants when used as a flavouring or food additive or health product or beverage or juice or coloring.

Plant parts contemplated by the present invention includes, but is not limited to flowers, fruits, vegetables, nuts, roots, stems, leaves or seeds.

The extracts of the present invention may be derived from the plants or plant part or cells therefrom in a number of different ways including but not limited to chemical extraction or heat extraction or filtration or squeezing or pulverization.

The plant, plant part or cells therefrom or extract can be utilized in any number of different ways such as for the production of a flavouring (e.g. a food essence), a food additive (e.g. a stabilizer, a colorant) a health product (e.g. an antioxidant, a tablet) a beverage (e.g. wine, spirit, tea) or a juice (e.g. fruit juice) or coloring (e.g. food coloring, fabric coloring, dye, paint, tint).

A further aspect of the present invention is directed to recombinant forms of pH modulating or altering proteins. The recombinant forms of the enzyme will provide a source of material for research, for example, more active enzymes and may be useful in developing in vitro systems for production of colored compounds.

Still a further aspect of the present invention contemplates the use of the genetic sequences described herein in the manufacture of a genetic construct capable of expressing a pH modulating or altering protein or down-regulating an indigenous pH modulating protein in a plant.

The term genetic construct has been used interchangeably throughout the specification and claims with the terms “fusion molecule”, “recombinant molecule”, “recombinant nucleotide sequence”. A genetic construct may include a single nucleic acid molecule comprising a nucleotide sequence encoding a single protein or may contain multiple open reading frames encoding 2 or more proteins. It may also contain a promoter operably linked to 1 or more of the open reading frames.

Another aspect of the present invention is directed to a prokaryotic or eukaryotic organism carrying a genetic sequence encoding a pH modulating or altering proteins extrachromosomally in plasmid form.

The present invention further extends to a recombinant polypeptide comprising a sequence of amino acids substantially as set forth in SEQ ID NO: 2, 4 or 6 or an amino acid sequence having at least about 50% similarity to SEQ ID NO: 2, 4 or 6 or a derivative of said polypeptide.

How should rose SEQ be included here? SEQs 98 & 99.

A “recombinant polypeptide” means a polypeptide encoded by a nucleotide sequence introduced into a cell directly or indirectly by human intervention or into a parent or other relative or precursor of the cell. A recombinant polypeptide may also be made using cell-free, in vitro transcription systems. The term “recombinant polypeptide” includes an isolated polypeptide or when present in a cell or cell preparation. It may also be in a plant or parts of a plant regenerated from a cell which produces said polypeptide.

A “polypeptide” includes a peptide or protein and is encompassed by the term “enzyme”.

The recombinant polypeptide may also be a fusion molecule comprising two or more heterologous amino acid sequences.

Still yet another aspect of the present invention contemplates a pH modulating or altering nucleic acid sequence linked to a nucleic acid sequence involved in modulating or altering the anthocyanin pathway.

pH4 is a member of the MYB family of transcription factors that is expressed in the petal epidermis and that can physically interact with AN1 and JAF13. This indicates that AN1 is present in at least two distinct transcription complexes. One complex includes pH4 and activates a set of unknown target genes involved in vacuolar acidification, whereas another (pH4-independent) complex activates the structural anthocyanin genes.

The present invention is further described by the following non-limiting Examples.

### Example 1

### General Methods

In general, the methods followed were as described in Sambrook et al. (1989, supra) or Sambrook and Russell, Molecular Cloning: A Laboratory Manual 3^{rd} edition, Cold Spring Harbor Laboratories, Cold Spring Harbor, N.Y., USA, 2001 or Plant Molecular Biology Manual (2^{nd} edition), Gelvin and Schilperoot (eds), Kluwer Academic Publisher, The Netherlands, 1994 or Plant Molecular Biology Labfax, Croy (ed), Bios scientific Publishers, Oxford, UK, 1993.

### Stages of Flower Development

Petunia hybrida cv. M1×V30 flowers were harvested at developmental stages defined as follows:

Stage 1: Unpigmented flower bud (less than 10 mm in length)
Stage 2: Unpigmented flower bud (10 to 20 mm in length)
Stage 3: Lightly pigmented closed flower bud (20 to 27 mm in length)
Stage 4: Pigmented closed flower bud (27 to 35 mm in length)
Stage 5: Fully pigmented closed flower bud (35 to 45 mm in length)
Stage 6: Fully pigmented bud with emerging corolla (45 to 55 mm in length)
Stage 7: Fully opened flower (55 to 60 mm in length)

Other petunia cultivars (such as R27 and W115) were grouped into similar developmental stages as described above however the overall lengths of the buds varied between cultivars.

### Plant Material

The Petunia hybrida lines used in the cDNA-AFLP screening were R27 (wild-type (wt)), W225 (an1, frame-shift mutation in R27 background), R144 ph3-V2068 transposon insertion in PH3 in R27 background), R147 (ph4-X2058 transposon insertion in PH4 in R27 background) and R153 (ph5 transposon insertion in PH5 crossed into a R27 background). All lines have genetically identical background and to diminish differences in environmental conditions which could lead to differences in transcript levels, the plants were grown in a greenhouse adjacent to each other.

The Petunia hybrida line M1×V30 used in transformations experiments was an F1 hybrid of M1 (AN1, AN2, AN4, PH4, PPM1, PPM2) crossed with line V30 (AN1, AN2, AN4, PH4, PPM1, PPM2). Flowers of M1×V30 are red-violet and generally accumulate anthocyanins based upon malvidin and low levels of the flavonol quercetin.

Petunia hybrida Transformations)

As described in Holton et al. (Nature, 366: 276-279, 1993) or Brugliera et al, (Plant J. 5, 81-92, 1994) or de Vetten N et al (Genes and Development 11: 1422-1434, 1997) or by any other method well known in the art.

Preparation of petunia R27 Petal cDNA Library

A petunia petal cDNA library was prepared from R27 petals using standard methods as described in Holton et al. (1993, supra) or Brugliera et al, (1994, supra) or de Vetten N et al (1997, supra).

### Transient Assays

Transient expression assays were performed by particle bombardment of petunia petals as described previously (de Vetten et al, supra; Quattrocchio et al, Plant J. 13, 475-488, 1998).

### pH Assay.

The pH of petal extracts was measured by grinding the petal limbs of two corollas in 6 mL distilled water. The pH was measured directly (within 1 min) with a normal pH electrode, to avoid that atmospheric CO₂ would alter pH of the extract

### HPLC and TLC Analysis

HPLC analysis was as described in de Vetten et al. (Plant Cell 11(8):1433-1444, 1999). TLC analysis was as described in van Houwelingen et al, (Plant J. 13(1): 39-50, 1998)

### Analysis of Nucleotide and Predicted Amino Acid Sequences

Nucleotide and predicted amino acid sequences were analyzed with the program Geneworks (Intelligenetics, Mountain View, Calif.). Multiple sequence alignments were produced with a web-based version of the program ClustalW (http://dot.imgen.bcm.tmc.edu:9331/multi-align/multi-align.html) using defaults settings (Matrix=blossom; GAPOPEN=0, GAPEXT=0, GAPDIST=8, MAXDIV=40). The phylogenetic tree was build with PHYLIP (bootstrap count=1000) via the same website, and visualized with Treeviewer version 1.6.6 (http://taxonomy.zoology.gla.ac.uk/rod/rod.html)

### RNA Isolation and RT-PCR

RNA isolation and RT-PCR analysis were carried out as described by de Vetten et al, (1997, supra). Rapid amplification of cDNA (3′) ends (RACE) was done as described by Frohman et al. (PNAS 85: 8998-9002, 1988).

### Example 2

### Transcript Profile Analysis

A combination of cDNA-AFLP and microarray analysis were utilised in order to identify transcripts that were downregulated in an1⁻, ph3⁻ and ph4⁻ mutants. A summary of results is shown in Table 3

TABLE 3
Trancripts identified by cDNA-AFLP or microarray analysis
that are down regulated in an1⁻, ph3⁻ and ph4⁻ mutants
and found at wild-type levels in ph2⁻ and ph5⁻ mutants

| | Size | | Down | |
|---|---|---|---|---|
| Name | (bp) | Normal | regulated | NCBI Blast search |
| CAC 4.4 | 116 | wt, ph2, ph5 | an1, ph3, ph4 | No significant similarity |
| CAC 5.6 | 250 | wt, ph2, ph5 | an1, ph3, ph4 | putative outer membrane protein |
| CAC 7.0 | 300 | wt, ph2, ph5 | an1, ph3, ph4 | No significant similarity |
| CAC 7.4 | 150 | wt, ph2, ph5 | an1, ph3, ph4 | No significant similarity |
| CAC 7.5 | 170 | wt, ph2, ph5 | an1, ph3, ph4 | putative PM-type protein |
| CAC 8.3 | 150 | wt, ph2, ph5 | an1, ph3, ph4 | No significant similarity |
| CAC 8.9 | 252 | wt, ph2, ph5 | an1, ph3, ph4 | PREG1 like neg. regulator |
| CAC 10.6 | 181 | wt, ph2, ph5 | an1, ph3, ph4 | putative phosphatidylinositol |
| | | | | kinase |
| CAC 12.1 | 71 | TBD | TBD | TBD |
| CAC 12.3 | 803 | wt, ph2, ph5 | an1, ph3, ph4 | 3′-5′ exonuclease containing |
| | | | | protein |
| CAC 13.4 | 126 | wt, ph2, ph5 | an1, ph3, ph4 | unknown protein |
| CAC 13.10 | 452 | wt, ph2, ph5 | an1, ph3, ph4 | membrane transporter like protein |
| CAC 14.2 | 1276 | wt, ph2, ph5 | an1, ph3, ph4 | no long ORF |
| CAC 14.3 | 1312 | wt, ph2, ph5 | an1, ph3, ph4 | putative SPFH domain containing |
| | | | | protein |
| CAC 14.4 | TBD | TBD | TBD | TBD |
| CAC 16.1 | 188 | wt, ph2, ph5 | an1, ph3, ph4 | No significant similarity |
| CAC 16.2 | 1440 | wt, ph2, ph5 | an1, ph3, ph4 | no long ORF |
| CAC 16.5 | 1025 | wt, ph2, ph5 | an1, ph3, ph4 | cysteine proteinase |
| MAC F55 | full | wt, ph2, ph5 | an1, ph3, ph4 | Plasma membrane ATPase |
| | length | | | |
| MAC 1D2 | 1164 | wt, ph2, ph5 | an1, ph3, ph4 | putative myosin protein |
| MAC 9F1 | 956 | wt, ph2, ph5 | an1, ph3, ph4 | unknown protein |
| MAC 10F12 | TBD | TBD | TBD | TBD |

ORF = open reading frame
TBD = to be done
CAC = transcript identified using cDNA-AFLP
MAC = transcript identified using microarray
NCBI- Blast search = Any similarities to known sequences were discovered by using a BLAST search (Altschul et al. Nucl. Acids Res. 25: 3389-3402, 1997) on the National Center for Biotechnology Information (NCBI) website (as of February 2005).

### Example 3

### Description of cDNA-AFLP

Using 256 primer combinations of MseI+NN/EcoRI+NN, around 20,000 fragments were analysed which covered around 25% of total transcripts. 80 fragments were isolated from the gel and 20 were further characterised by RT-PCR of total RNA isolated from petunia mutant lines including wild-type and an1, ph2, ph3, ph4, ph5 mutants. Sixteen of these fragments (see Table 3) were confirmed as being down-regulated in an1, ph3 and ph4 petunia lines compared to their expression levels in wild-type, ph2 and ph5 petunia lines.

RNA Isolation and cDNA Synthesis

The petunia lines R7 (wt), W225 (an1⁻), R144 (ph3⁻), R147 (ph4⁻) and R153 (ph5⁻) were used in the cDNA-AFLP screening. Around 25 to 30 flower buds (flower developmental stage 5, 6) were harvested from each petunia line and stored at −70° C. Total RNA was extracted from 10 corollas according to Logemann et al. (Anal Biochem. 163(1):16-20, 1987). PolyA⁺ RNA was then isolated from 500 micrograms of total RNA using oligo(dT) coupled to magnetic beads according to the PolyATract® System (PROMEGA) protocol. One microgram of polyA⁺ RNA was then used for synthesizing double stranded (ds) cDNA using the GIBCO-BRL Superscript II system. After synthesis of ds cDNAs, the cDNAs were phenol extracted (Sambrook et al, 2001, supra) and the cDNA precipitated with the addition of salt and ethanol. The DNA pellet was then resuspended in 30 μL of distilled water.

### Template Preparation

Restriction endonuclaseses MseI (digests a 4 base recognition sequence) and EcoRI (digests a 6 base recognition sequence) were used for the template preparation for cDNA-AFLP analysis. The cDNAs were digested with both restriction endonucleases in combination with ligation of adapters (Mse A1 (SEQ ID NO: 7) and Mse A2 (SEQ ID NO: 8)) annealed to each other and EcoA1 (SEQ ID NO: 14) and EcoA2 (SEQ ID NO: 15) also annealed to each other to form respectively a PCR adaptor for the MseI site and one for the EcoRI site) to the MseI and EcoRI ends. Each “restriction-ligation” reaction was performed in a total volume of 50 μL which included 24 μL ds cDNA, 10 μL 5×RL buffer (50 mM Tris HAc pH7.5, 50 mM MgAc, 250 mM KAc, 25 mM DTT, 250 μg/μL BSA), 0.1 μL 100 mM ATP, 5 units MseI (New England Biolabs), 5 units EcoRI (New England Biolabs), 50 pmol MseI adapter (Mse A1 and Mse A2) (SEQ ID NO: 7 and 8) and 50 pmol EcoRI adapter (EcoA1 and EcoA2) (SEQ ID NO: 14 and 15). The adapters had previously been boiled for 2 minutes and then slowly allowed to cool to room temperature prior to their addition to the reaction. The “restriction-ligation” reaction was incubated for 4 hours at 37° C.

### Amplification

Prior to amplification, cDNA templates were diluted 10-fold in water and then a volume of 10 μL was used in the first, non-radioactive, PCR amplification step with one nucleotide selective extension (EcoRI+N, MseI+N) primers (SEQ ID NO: 10 to 13 and 16 to 19))(see Table 4) in a touch-down PCR program. The PCR cycle included a 94° C. denaturation step followed by annealing step of 30 seconds at temperatures starting at 65° C. and reducing in 0.7° C. increments down to 56° C. over 17 cycles followed by 18 cycles of 56° C. for 30 sec and finally an elongation step at 72° C. for 1 min. Eight microliters of the products from this first PCR were electrophoresced through a 1% agarose gel and the expected DNA smear between 200 and 750 bp was detected. Subsequently, 0.5 μL of these products were used as template in a second “hot” PCR using 2 nucleotide extension (EcoRI+NN, MseI+NN) primers (SEQ ID NO. 20 to 51) (see Table 5) in standard PCR conditions with a touch-down PCR program as described previously. The EcoRI primers in the second PCR were radio-labeled with ³³P in a reaction which included 50 ng primer, 5 μL 10× T4 kinase buffer, 10 μL ³³P-CTP, 24 μL water and 9 units T4 polynucleotide kinase. The reaction was incubated for 1 hour at 37° C., followed by inactivation of the T4 kinase by treatment at 65° C. for 10 minutes.

TABLE 4
Primers used in the cDNA-AFLP analysis

| SEQ | | | |
|---|---|---|---|
| ID | Primer | Primer | |
| NO. | No. | name | Primer sequence (5′ to 3′) |
| 7 | 701 | Mse A1 | GAC GAT GAG TCC TGA G |
| 8 | 702 | Mse A2 | TAC TCA GGA CTC AT |
| 9 | 703 | mse + 0 | GAC GAT GAG TCC TGA GTA A |
| 10 | 704 | Mse + A | GAC GAT GAG TCC TGA GTA AA |
| 11 | 705 | Mse + C | GAC GAT GAG TCC TGA GTA AC |
| 12 | 706 | Mse + G | GAC GAT GAG TCC TGA GTA AG |
| 13 | 707 | Mse + T | GAC GAT GAG TCC TGA GTA AT |
| 14 | 724 | EcoA1 | GTG ATA TCT CCA CTG ACG T |
| 15 | 725 | EcoA2 | CTC GTA GAC TGC GTA CC |
| 16 | 726 | Eco + A | AAT TGG TAC GCA GTC |
| 17 | 727 | Eco + G | AGA CTG CGT ACC AAT TCA |
| 18 | 728 | Eco + G | AGA CTG CGT ACC AAT TCC |
| 19 | 729 | Eco + T | AGA CTG CGT ACC AAT TCG |

TABLE 5

Primers with 2 nucleotide extensions used in
the cDNA-AFLP analysis

| SEQ | | | |
|---|---|---|---|
| ID | Primer | Primer | |
| NO. | No. | name | Primer sequence (5′ to 3′) |
| 20 | 708 | Mse + AA | GAT GAG TCC TGA GTA AAA |
| 21 | 709 | Mse + AC | GAT GAG TCC TGA GTA AAC |
| 22 | 710 | Mse + AG | GAT GAG TCC TGA GTA AAG |
| 23 | 711 | Mse + AT | GAT GAG TCC TGA GTA AAT |
| 24 | 712 | Mse + CA | GAT GAG TCC TGA GTA ACA |
| 25 | 713 | Mse + CC | GAT GAG TCC TGA GTA ACC |
| 26 | 714 | Mse + CG | GAT GAG TCC TGA GTA ACG |
| 27 | 715 | Mse + CT | GAT GAG TCC TGA GTA ACT |
| 28 | 716 | Mse + GA | GAT GAG TCC TGA GTA AGA |
| 29 | 717 | Mse + GC | GAT GAG TCC TGA GTA AGC |
| 30 | 718 | Mse + GG | GAT GAG TCC TGA GTA AGG |
| 31 | 719 | Mse + GT | GAT GAG TCC TGA GTA AGT |
| 32 | 720 | Mse + TA | GAT GAG TCC TGA GTA ATA |
| 33 | 721 | Mse + TC | GAT GAG TCC TGA GTA ATC |
| 34 | 722 | Mse + TG | GAT GAG TCC TGA GTA ATG |
| 35 | 723 | Mse + TT | GAT GAG TCC TGA GTA ATT |
| 36 | 730 | Eco + AA | GAC TGC GTA CCA ATT CAA |
| 37 | 731 | Eco + AC | GAC TGC GTA CCA ATT CAC |
| 38 | 732 | Eco + AG | GAC TGC GTA CCA ATT CAG |
| 39 | 733 | Eco + AT | GAC TGC GTA CCA ATT CAT |
| 40 | 734 | Eco + CA | GAC TGC GTA CCA ATT CCA |
| 41 | 735 | Eco + CC | GAC TGC GTA CCA ATT CCC |
| 42 | 736 | Eco + CG | GAC TGC GTA CCA ATT CCG |
| 43 | 737 | Eco + CT | GAC TGC GTA CCA ATT CCT |
| 44 | 738 | Eco + GA | GAC TGC GTA CCA ATT CGA |
| 45 | 739 | Eco + GC | GAC TGC GTA CCA ATT CGC |
| 46 | 740 | Eco + GG | GAC TGC GTA CCA ATT CGG |
| 47 | 741 | Eco + GT | GAC TGC GTA CCA ATT CGT |
| 48 | 742 | Eco + TA | GAC TGC GTA CCA ATT CTA |
| 49 | 743 | Eco + TC | GAC TGC GTA CCA ATT CTC |
| 50 | 744 | Eco + TG | GAC TGC GTA CCA ATT CTG |
| 51 | 745 | Eco + TT | GAC TGC GTA CCA ATT CTT |

### Analysis of PCR Products:

The reaction products were analyzed by electrophorescing through a 5% denaturing polyacrylamide gel. After electrophoresis the gels were dried on a slab gel dryer and then exposed overnight. The radiolabelled signals of the reaction products were then detected using a Phosphor imager (Molecular Dynamics, Sunnyvale, Calif., USA).

In summary using 256 primer combinations of MseI+NN/EcoRI+NN, around 20,000 fragments were analysed which covered around 25% of total transcripts. 80 fragments were isolated from the gel and 20 were further characterised by RT-PCR of total RNA isolated from petunia mutant lines including wild-type and an1, ph2, ph3, ph4, ph5 mutants. Sixteen of these CAC fragments (see Table 3) were confirmed as being down-regulated in an1, ph3 and ph4 petunia lines compared to their expression levels in wild-type, ph2 and ph5 petunia lines. A summary of the CAC fragments and their respective sizes along with detected sequence similarities to known sequences is shown in Table 6.

TABLE 6
A summary of fragments isolated by cDNA-AFLP that are down-regulated
in an1, ph3 and ph4 petunia lines compared to their expression
levels in wild-type, ph2 and ph5 petunia lines.

| | | | Similarity | | |
|---|---|---|---|---|---|
| Fragment | Further info | BLASTx result | E-value | Fragment size | |
| CAC 4.4 | | NSS | — | 114 | bp |
| CAC 5.6 | | Putative membrane prot. | 1 | 250 | bp |
| CAC 6.6 | | NSS | — | 191 | bp |
| CAC 7.0 | | ESTc74501(rice)/lipid transfer | 0.021/0.17 | 279 | |
| | | protein (A. th) | | | |
| CAC 7.4 | | Putative senescence ass. prot. | 1 × E⁻¹⁹ | 350 | |
| CAC 7.5 | | Putative plasma membrane prot. | 0.2 | 543 | bp |
| CAC 8.3 | | No sequence | — | — | |
| CAC 8.8 | | Glycolate oxidase | 0.015 | 95 | bp |
| CAC 8.9 | | PREG1-like negative regulator | 1 × E⁻²⁹ | 245 | bp |
| CAC10.6 | | Put. phosphatidyl kinase | 1 × E⁻¹¹ | 181 | bp |
| CAC 12.1 | | NSS | — | 71 | bp |
| CAC 12.3 | | 3contains 3′-5′exonucl. domain | 2 × E⁻⁵ | 845 | bp |
| CAC 13.4 | | Unknown prot. (A. th.) | 2 × E⁻¹⁰ | 124 | bp |
| CAC 13.10 | | Membrane transporter | 1 × E⁻¹⁰ | 346 | bp |
| CAC 14.2 | | Same than 16.2 | — | 1261 | bp |
| CAC 14.3 | | Putative SPFH protein | 1 × E−137 | 1312 | bp |
| CAC 14.4 | | No sequence data | — | — | |
| CAC 16.1 | | Histone H2B-like prot. (TAIR) | 0.0077 | 87 | bP |
| CAC 16.2 | | No long ORF | — | 1405 | bp |
| CAC 16.4 | | No sequence data | — | — | |
| CAC 16.5 | | Cystein proteinase | 2 × E⁻⁵⁰ | 1169 | bp |
| CAC 13.2 | Only down in | Anthocyanins 3-O- | 6 × E⁻¹⁰ | 215 | bp |
| | an1 mutants | glucosyltransferase | | | |
| CAC 8.11 | Up in ph3, ph4 | Hypothetical Protein AF420410 | 1 × E⁻¹⁸ | 255 | bp |
| | and an1 mutants | | | | |
| CAC 4.5 | Only down in | Anthocyanins 5-O- | 1 × E⁻²¹ | 251 | bp |
| | an1 mutants | glucosyltransferase | | | |

Similarity E-value = a parameter generated by a BLASTX search that indicates the relative identity to an aligned sequence. The closer to 0 the E-value is the more significant the match
NSS = no sequence similarity

### Example 4

### Micro Array Analysis

For the micro-array hybridization, petal tissue of developmental stage 5 of both wildtype (R27) and an1⁻ mutant line (W225) was used to isolate polyA⁺ RNA according to protocol of the supplier (polyATtract mRNA Isolation System III, Promega). Microarrays were prepared and hybridised using conditions described by Verdonk et al. (Phytochemistry 62: 997-1008, 2003).

### Description of Microarray

Of 1415 ESTs spotted onto microarrays, 9 ESTs were found to be down-regulated by more than 10-fold in the an1 mutant petunia line (W225). Five of these sequences represented genes previously isolated and characterised (see Table 7). Four ESTs were further characterised by RT-PCR of total RNA isolated from petunia mutant lines including wild-type and an1, ph2, ph3, ph4, ph5 mutants. Two of these ESTs (MAC F55 and MAC 9F1) were confirmed as being down-regulated in an1 petunia lines.

TABLE 7
Clones identified in the microarray screen that showed
50 to 100 times downregulation in an1 mutants.

| | | | Similarity | Fragment |
|---|---|---|---|---|
| Fragment | Further info | BLASTx result | E-value | size |
| MAC F55 | | Plasma ATP-ase | 1 × E⁻³⁹ | 2850 bp |
| MAC ID12 | | Putative myosin | 2 × E⁻⁴⁸ | 1511 bp |
| | | heavy chain | | |
| MAC 9F1 | | A. thaliana | 1 × E⁻¹⁶ | 687 bp |
| | | At2g17710 | | |
| | | expressed prot. | | |
| MAC C90 | | No sequence data | — | — |
| MAC 10F12 | | TBD | TBD | TBD |
| MAC M33 | Already known | Cyt. b5 like | 0 | Full size |
| | AN1 target | | | cDNA |
| MAC | Already known | Petunia DFR-A | 0 | Full size |
| DFRA | AN1 target | | | cDNA |
| MAC Rt | Already known | Petunia RT | 0 | Full size |
| | AN1 target | | | cDNA |
| MAC AN9 | Already known | Petunia GST | 0 | Full size |
| | AN1 target | | | cDNA |

Several more clones show a lower level of down regulation and could be considered in a second round of analysis.

The expression pattern and genetic control was determined for several of these genes by RT PCR in different petunia tissues and in flowers of wild type and mutant plants. The majority of these genes show higher expression in petals than in other parts of the plant and the expression studies in the mutants confirmed the pattern previously seen by transcript profiling.

### Example 5

### Construction of RNAi Constructs for Expression in Petunia

In order to assess the role of these genes in the acidification of the vacuolar lumen in flower epidermal cells inverted repeat constructs of each gene were or are expressed in wild-type petunia plants with the aim of silencing the endogenous gene.

To date down regulation of three genes has resulted in a change in flower color with a concomitant change in vacuolar pH. These include MAC F55 (PPM1) (SEQ ID NO: 1), MAC 9F1 (SEQ ID NO: 3) and CAC 16.5 (SEQ ID NO: 5).

### Down Regulation of MAC F55 (PPM1)

The MAC F55 clone codes for a plasma membrane ATPase (PPM1, Petunia Plasma Membrane ATPase 1) (SEQ ID NO: 1) and has a relatively high sequence identity with ATPase genes already isolated. However, alignment of the different members of the ATPase gene family, show that PPM1 groups together with AHA10 from Arabidopsis and PMA9 from Tobacco in the class III (Arango et al. Planta, 216: 335-365, 2003). These proteins all diverge from the other plasma ATPases in the C terminal part, which represents the site of interaction with 14.3.3 factors regulating the activity of the pump. Cellular localization and function have never been defined for any member of this group, leaving open the possibility that PPM1 resides in other cellular membranes than the plasma membrane. A recent publication by Baxter et al, (PNAS, 102: 2649-2654, 2005) describes analysis of Arabidopsis AHA10 mutants. AHA 10 was described as having a specific effect on proanthocyanidin and vacuole biogenesis. The aha10 mutants characterised had decreased levels of proanthocyanidins in their seed coats and the seed coat endothelial cells displayed many small vacuoles rather than one central vacuole as observed in wild-type seeds.

In order to assess the role of PPM1 gene in the acidification of the vacuolar lumen in flower epidermal cells, wild type petunia plants (V30×M1) were transformed with two inverted repeat constructs: a 233 bp inverted repeat spanning from nucleotide 2937 to nucleotide 3170 of the PPM1 full size cDNA (SEQ ID NO: 1) and a 499 bp inverted repeat spanning from nucleotide 2671 to nucleotide 3170 of the PPM1 full size cDNA (SEQ ID NO: 1), both under the control of the CaMV 35S promoter.

### Inverted Repeat Constructs (Gateway)

A P. hybrida R27 petal cDNA library was hybridized with ³²P-labelled fragments of PPM1. The PPM1 fragment was generated using PCR amplification with first stand cDNA from RNA isolated from petunia petals as template and the primers #1702 (SEQ ID NO: 52) and #1703 (SEQ ID NO: 53). The full length PPM1 sequence was obtained using a double 5′ Rapid Amplification of cDNA (5′/3′-RACE KIT 2^{ND} generation, Roche, USA) according to the manufacturer's protocols. Primers #1703 (SEQ ID NO: 53), #1742 (SEQ ID NO: 55) and #1832 (SEQ ID NO: 61) were used for the first 5′-RACE whilst primers #1789 (SEQ ID NO: 58), #1812 (SEQ ID NO: 59) and #1831 (SEQ ID NO: 60) were used for the second 5′-RACE.

PCR conditions in all amplifications was as follows: 96° C., 30 seconds, 65° C., 30 seconds and 72° C. for 3 minutes, 32 cycles (T3 thermocycler, Biometra).

TABLE 8
Primers used in amplification of PPM1 fragments.

| SEQ | | | |
|---|---|---|---|
| ID | Primer | | |
| NO: | No. | Direction | Sequence 5′ to 3′ |
| 52 | 1702 | Forward | GGACCTTAACAAAATTCAAACAG |
| 53 | 1703 | reverse | AAATTAATGAATGATATGAGG |
| 54 | 1741 | Forward | TGAAGAAATGTCATCAGCCG |
| 55 | 1742 | reverse | GTTCAGCAATCATAGATGGC |
| 56 | 1750 | Forward | GCTCTGACTGGAGAAGCCTGG |
| 57 | 1788 | Forward | CCAAGAGAAGCAACAGATAGCTGCAA |
| 58 | 1789 | reverse | TTGCAGCTATCTGTTGCTTCTCTTGG |
| 59 | 1812 | reverse | GAATCAATGTAAGTGATTGCAGTCCG |
| 60 | 1831 | reverse | AACTGATAGGACTGTTGGCATAGC |
| 61 | 1832 | reverse | GCTGGTGCATCATTTACTCCATC |
| 62 | 1847 | Forward | ATGGCCGAAGATCTGGAGAGACC |
| 63 | 1848 | reverse | CTGCAGGGATGATATCACCAAGC |
| 64 | 1861 | Forward | CTGATAATAGCAATCCTAAATGATGG |
| 65 | 1864 | Forward | CGGAATTCATGGCCGAAGATCTGGAGAGACCTTTAC |
| 66 | 1885 | reverse | CCCGGGCTTCTCCAGTCAGAGCATATCAAACAGCAA |
| 67 | 1886 | Forward | AAGAATTCGTTTGTTATGCTCTGACTGGAGA |
| 68 | 1956 | reverse | GACTGCGGGTAACAAATATTAGCG |
| 69 | 2035 | Forward | GCAAATATCAGGGAAGTGCATTTCC |
| 70 | 2037 | Forward | CGGAATTCTCGCAAATATCAGGGAAGTGCATTTCCTT |
| 71 | 2038 | reverse | TTATGAATCAATGTAAGTGATTGCAGTCCG |
| 72 | 2039 | Forward | TAGCCCATGGCCGAAGATCTGGAGAGACC |
| 73 | 2040 | reverse | CATGAGCCATGGACAAACTGTATGAGCTGTTTG |
| 74 | 2073 | Forward | GCTTGCTGATCCAAAGGAGGCACGT |
| 75 | 2075 | reverse | GTAAGGATTCCCCAGTAAGAGC |
| 76 | 2078 | reverse | CGGGATCCTGGAGCCAGAAGTTTGTTATAGGAGG |
| 77 | 2123 | reverse | GGTCTTGGAGATGGTTTAACCC |
| 78 | 2124 | Forward | GCTGCTAGGAGTGCTGGTGATCTTG |
| 79 | 2196 | reverse | GCATGATACAATGTCCTAGATTCACTTC |
| 80 | 2270 | Forward | CTAACCATGGCCGAAGACCTGGAGAGACCT |
| 81 | 2271 | reverse | GTTTGATCAGACGTCACATGTCTCCAAACTGTATGAGCTGTTTGA |

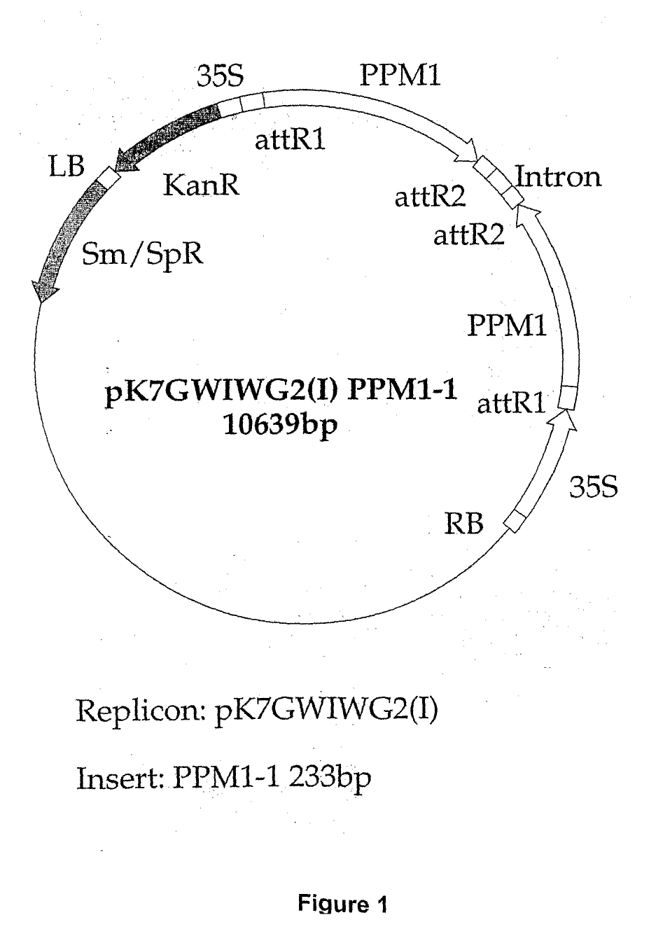

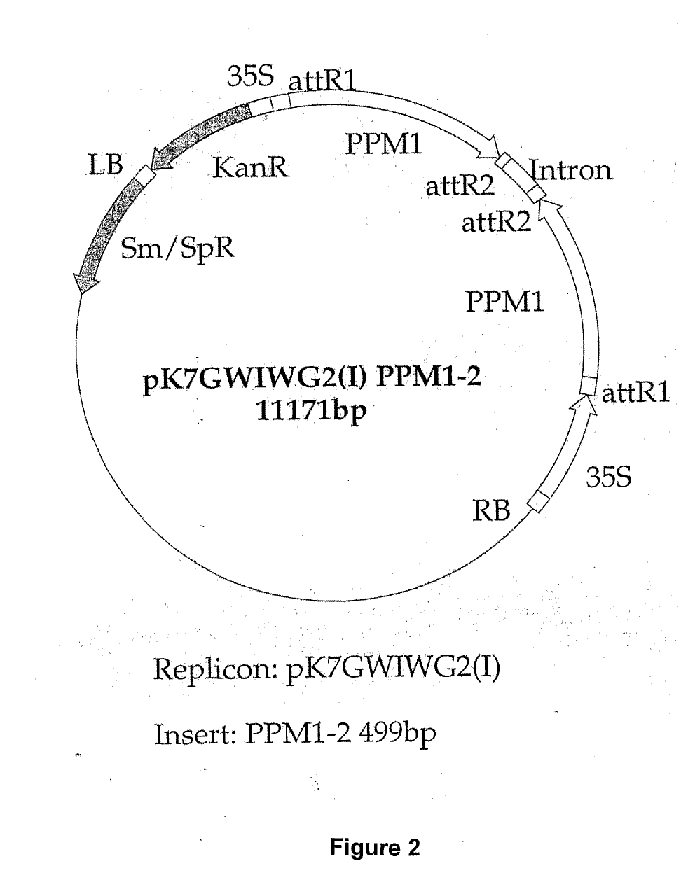

Two PPM1 cDNA fragments (A and B) were amplified using the following primers: A, #1703 (SEQ ID NO: 53) and # 1702 (SEQ ID NO: 52) and B, #1703 (SEQ ID NO: 53) and #1750 (SEQ ID NO: 56). The PCR products were then ligated into the vector pGemt-easy (Promega). Clones containing the correct insert were selected by PCR, digested with EcoRI and subsequently cloned into the EcoRI restriction site of the entry vector pDONR207(1) of the Gateway system (INVITROGEN). Using the Gateway LR recombination reaction (INVITROGEN), the inserts were translocated into pK7GWIWG2(I) and transformed into competent E. coli DH5α cells. With the primer combinations 35S promoter (#27) together with the pK7GWIWG2(I) intron reverse primer (#1777), and 35S terminator (#629) together with the intron forward primer (#1778) clones containing the insert in an inverted repeat arrangement were selected. Subsequently, these clones, pK7GWIWG2 (I) PPM1-1 (FIG. 1) and pK7GWIWG2 (I) PPM1-2, (FIG. 2) were introduced into Agrobacterium tumefaciens by electroporation and transfected into petunia via leaf disk transformation. Transformed plants were selected on MS plates containing 250 microgram/ml of kanamycin, and after rooting, were grown in normal greenhouse conditions.

Of the 6 transgenic plants produced using p K7GWIWG2 (I) PPM1-1, 6 resulted in a change in flower color from red to purple/blue. Of the 3 transgenic plants produced using p K7GWIWG2 (I) PPM1-2, 3 resulted in a change in flower color from red to purple/blue. The changes in color correlated with silencing of the endogenous PPM1 transcript and a pH increase of the crude flower extract of about 0.5 units. No effect was detectable on the amount and type of anthocyanin pigment accumulated in the flowers of the silenced plants as determined by TLC and HPLC.

Petunia plants mutated in different petunia pH loci as well as those transgenic plants showing silencing of PPM1, still express another member of the plasma membrane ATPase family from Petunia namely, PPM2.

PPM2 shows high homology with class II of plasma ATPase proteins containing PMA4 from Nicotiana and AHA2 from Arabidopsis for which plasma membrane localization in plant cells has been shown, as well as the capability of complementing pmp1 mutants in yeast and their regulation by 14.3.3 proteins (Jahn et al, JBC, 277, 6353-6358, 2002).

TABLE 9

Primers used in amplification of PPM2
fragments.

| SEQ | | | |
|---|---|---|---|
| ID | | | |
| NO: | PPM2 | Direction | Sequence (5′ to 3′) |
| 82 | 1969 | forward | CTTGTTGACAGCACCAACAATG |
| 83 | 1970 | reverse | CAAGGATCTATCGACACTCAACTTG |

The PPM-1 gene is intriguing because the possible involvement of a P-type ATPase in vacuolar acidification has never been proposed before. From preliminary analysis of PPM1 expression in Petunia it was found that the gene is specifically expressed in the flower limb (nowhere else in the plant). Because petunia flowers mutated in AN1, PH3 or PH4 do not show any expression of PPM-1, and still look healthy, it is tempting to think that the function of this specific gene is confined to the control of the vacuole environment, while it does not contribute to the regulation of the cytosolic pH. It is also possible that other members of the P-ATPase family are expressed in these same cells and control the proton gradient through the plasma membrane.

A question of considerable significance concerns the cellular localization of this protein. P-ATPases are membrane associated proteins but in this specific case it was not expected that the PPM-1 protein to be localized on the plasma membrane as this would not explain its contribution to vacuolar pH control. A GFP fusion of the full-size PPM-1 cDNA was expressed in petunia cells (transient expression in flowers via particle bombardment) and its localization was visualized by confocal microscopy. The different cellular compartment and vacuolar types are identified by marker GFP fusions (Di Sansebastiano et al, Plant Physiology, 126, 78-86, 2001). The PPM-1 protein appeared to be localized on the tonoplast or in vesicles that later fuse to the central vacuole of the flower epidermal cells, which opens a new view of the role of these proteins in cellular homeostasis.

The capability of a PPM-1 expression construct to complement the yeast Pma1 mutant missing the endogenous P-ATPase activity is tested to make sure that PPM-1 encoded proteins has indeed P-ATPase activity.

Further studies on the role of PPM-1 in the pathway leading to flower vacuole acidification will suggest studies on how the activity of this class of P-ATPases is regulated. As already mentioned, nothing is known about the function and regulation of class III P-ATPases in plants. Although the protein sequences are overall very homologous to those of other P-ATPases, these proteins have a different sequence in the C-terminal tail that has been demonstrated to enable interaction with 14-3-3 proteins required for reaching a high state of activation (Arango et al, 2003, supra). This raises the question whether P-ATPases of this class interact with 14-3-3 regulators or not. A yeast two hybrid screening of a petunia corolla cDNA library was performed to look for proteins interacting with this part of PPM-1 and the purified PPM-1 protein was analysed for binding to 14-3-3 protein in vitro (overlay assay).

Phosphorylation of Thr947 has also been recognized as an important step in the regulation of the ATPase activity (Jahn et al, 2001, supra). The PH2 gene from petunia has been cloned and shown that this encodes a THr/Ser protein kinase and it was sought to be determined if PPM-1 is (directly or indirectly, e.g. via a cascade of Protein Kinases) the target of this kinase. To test this possibility, a full-size PPM-1 cDNA fused to a Hys-tag was expressed in wild type and in ph2⁻ petunia plants. The recombinant PPM-1 protein was purified from flower extracts using a nickel column, then visualised using SDS-PAGE and immunodetection with anti-ATPase and antiphosphothreonine antibodies. This therefore may assist in reconstructing a new small part of this pH-controlling pathway.

### Down Regulation of MAC 9F1, a Target Gene of AN1, PH3 and PH4 Essential for Vacuolar Acidification

The nucleotide and derived amino acid sequence of the clone MAC 9F1 (SEQ ID NO: 3 and 4, respectively) do not show clear homology with any identified nucleic acid sequence or protein of known function, respectively. However when inverted repeats of 9F1 were expressed in petunia wild-type plants the silencing of the 9F1 endogenous gene resulted in blue flowers with increased flower extract pH.

### Inverted Repeat Constructs (Gateway)

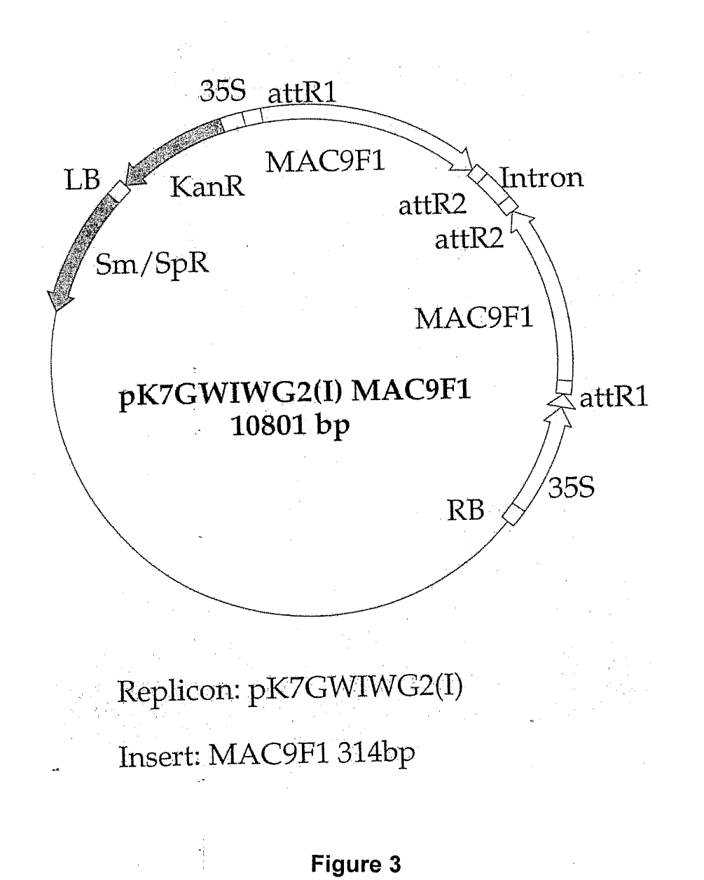

An inverted repeat construct, pK7GWIWG2(I) MAC9F1 (FIG. 3), of 9F1 was prepared using primers described in Table 10 and the Gateway system as described above. The inverted repeat 9F1 construct was introduced into Agrobacterium tumefaciens by electroporation and transfected into petunia via leaf disk transformation. Transformed plants were selected on MS plates containing 250 microgram/ml of kanamycin, and after rooting, were grown under normal greenhouse conditions.

Of 2 transgenic plants produced, 1 resulted in a change in flower color from red to purple/blue. The change in flower color correlated with silencing of the endogenous 9F1 gene and a pH increase of the crude flower extract of 0.5 units. No effect was detectable on the amount and type of anthocyanin pigment accumulated in the flowers of the silenced plants as determined by TLC and HPLC.

TABLE 10
Primers used in amplification of MAC9F1 fragments.

| SEQ | | | |
|---|---|---|---|
| ID | Primer | | |
| NO: | No. | Direction | Sequence 5′ to 3′ |
| 84 | 1706 | reverse | GTTCGCAAGCGCAATACTTAC |
| 85 | 1707 | forward | GGAATTCGGCACGAGGTCAC |
| 86 | 1743 | forward | AAGAGTAGCTGATCATGG |
| 87 | 1768 | forward | GATGAGGACATGAAGGAGCAAAGAG |
| 88 | 1876 | reverse | CTTCAGTCTTGCGTTTCTGCTTCC |
| 89 | 1877 | reverse | CTCCTGTTTTGTCAGGCTTGGTGC |
| 90 | 1878 | reverse | CGGCGGCGGTGGACTTGTCTTC |
| 91 | 2061 | reverse | GCTCTAGACTAGAATATGCCAAAAGTGGTTGCAAC |
| 92 | 2101 | forward | ATCGAATTCATGGCTGCACCAAGCCTAACAAAACAG |
| 93 | 2178 | reverse | ACCGCTCGAGCTAGAATATGCCAAAAGTGGTTGCAAC |

To gain more insight into the function of the small protein encoded by the 9F1 gene, cellular localization is identified by studying a GFP fusion in transient assay and possible interacting partners identified by yeast two hybrid screening of a cDNA library. An indication of the biochemical function of 9F1 could also come from the phenotype of plants overexpressing this gene.

The result of a BLAST search with this protein identifies a small family of proteins of which the two members with the highest homology to 9F1 come from Arabidopsis and rice. The characterization of an Arabidopsis knockout (KO) mutant for the 9F1 homologue might therefore be helpful.

### Down Regulation of CAC16.5

The nucleotide and derived amino acid sequence of the clone CAC16.5 is shown in SEQ ID NO: 5 and 6, respectively. The predicted amino acid sequence shows relatively high homology with Cysteine Proteases. The localization of these enzymes is typically vacuolar and their activity is dependent on relatively low environmental pH.

When a construct containing inverted repeats of CAC16.5 was introduced into petunia wild-type plants the silencing of the CAC16.5 endogenous gene surprisingly resulted in blue flowers with increased flower extract pH.

### Inverted Repeat Constructs (Gateway)

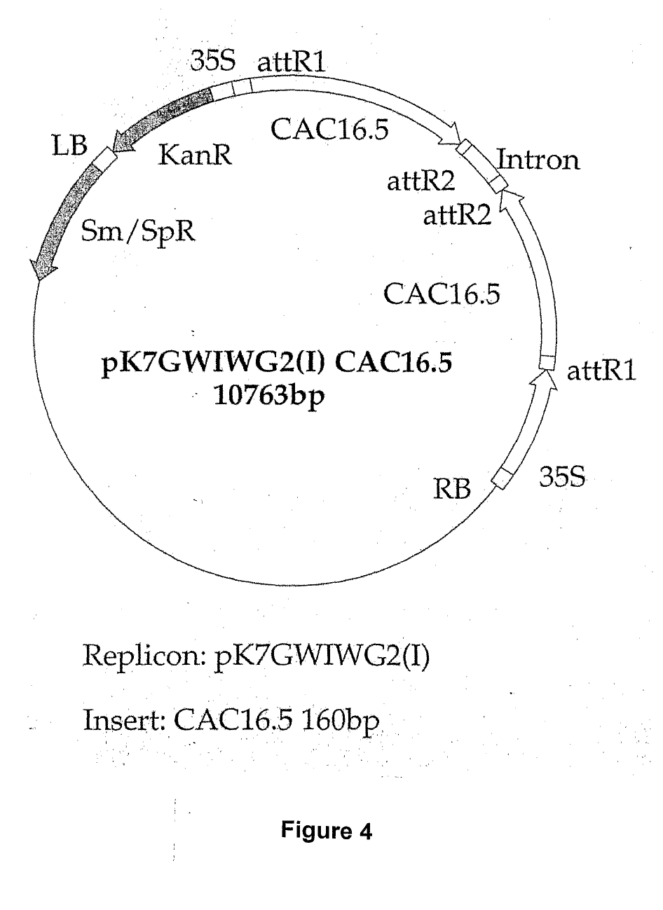

An inverted repeat construct, pK7GWIWG2(I) CAC16.5 (FIG. 4), of CAC16.5 was prepared using primers described in Table 11 and the Gateway system as described above.

The inverted repeat CAC16.5 construct was introduced into Agrobacterium tumefaciens by electroporation and transfected into petunia via leaf disk transformation. Transformed plants were selected on MS plates containing 250 microgram/ml of kanamycin, and after rooting, were grown in normal greenhouse conditions.

Of 4 transgenic plants produced, 3 resulted in a change in flower color from red to purple/blue. The change in flower color correlated with silencing of the endogenous CAC16.5 and a pH increase of the crude flower extract of 0.3 units. No effect was detectable on the amount and type of anthocyanin pigment accumulated in the flowers of the silenced plants as determined by TLC and HPLC.

TABLE 11

Primers used in amplification of CAC16.5
fragments.

| SEQ | | | |
|---|---|---|---|
| ID | Primer | | |
| NO: | No. | Direction | Sequence 5′ to 3′ |
| 94 | 1654 | reverse | CCTGTATATAGTTGGAAATCC |
| 95 | 1655 | forward | CAAGGCACTTGCAATATCACC |
| 96 | 1769 | reverse | GTAATGACATTCAAACAGCATCC |
| 97 | 1770 | forward | CTTCGTCGCCTCCTTATCCATCTCC |
| 98 | 1870 | reverse | GGATTATCAAGAATTCATGGGG |
| 99 | 1871 | reverse | GCCTCCTTATCCATCTCCAGCCC |

Because the function of Cysteine Proteases is the cleavage of a variety of other peptides, it would be interesting to identify the target of the proteolitic action of CAC16.5. To do this a “bait” plasmid is constructed for yeast two hybrid screening in which the Cys25 residue in the active site of the CAC16.5 gene is mutated. This will avoid the cleavage of the substrate when the two protein interact with each other and will allow to isolate the “prey” plasmid(s) containing the gene(s) that encodes for the target of CAC16.5. The characterization of the target of this proteolitic activity will help to further reconstruct the acidification pathway.

Detailed analysis of flowers from wild type, pH mutant and plants overexpressing regulators of the pH pathway has recently shown structural differences in the vacuoles of the epidermal cells (Quattrocchio et al, unpublished results). The most relevant difference involves the dimension and shape of the vacuoles in these cells and points towards a role of the PH genes in defining the height and width of vacuolar structure. Because the papillar shape of the cells in the corolla epidermis is peculiar to this tissue (to which this entire acidification pathway is restricted as shown by expression studies of the genes involved), it is proposed that the genes controlling acidity in the vacuolar lumen possibly also define the vacuole type (e.g. lytic or storage vacuole) and with it cell identity.

With this in mind, the pathway of events of AN1, PH3 and PH4 are dissected to understand if specific steps are related with the gaining of identity of the vacuole (and therefore of the cell) or the cell shape is simply a secondary effect of the internal pH of the vacuole compartment. The microscopic analysis of epidermal cells in flowers of plants silenced for different genes along the pH regulating pathway will provide an answer to this question and will possibly open a window on the mechanism of vacuolar diversification.

### Example 6

### Isolation of pH Modulating cDNAs from Other Species

Anthocyanins of an array of colors are produced in various species such as but not limited to Petunia sp., Plumbago sp., Vitis sp., Babiana stricta, Pinus sp., Picea sp., Larix sp., Phaseolus sp., Solanum sp., Vaccinium sp., Cyclamen sp., Iris sp., Pelargonium sp., Geranium sp., Pisum sp., Lathyrus sp., Clitoria sp., Catharanthus sp., Malvia sp., Mucuna sp., Vicia sp., Saintpaulia sp., Lagerstroemia sp., Tibouchina sp., Hypocalyptus sp., Rhododendron sp., Linum sp., Macroptilium sp., Hibiscus sp., Hydrangea sp., Ipomoea sp., Nicotiana sp., Cymbidium sp., Millettia sp., Hedysarum sp., Lespedeza sp., Antigonon sp., Pisum sp., Begonia sp., Centaurea sp., Commelina sp., Rosa sp., Dianthus sp. (carnation), Chrysanthemum sp. (chrysanthemums), Gerbera sp., Gentiana sp. Torenia sp., Nierembergia sp, Liatrus sp. etc.

It is expected that a number of these plants contain pH modulating sequences and that down regulation of these pH modulating sequences will result in a change in flower color.

Detection of Putative pH-Modulating Sequences in Oilier Plant Species

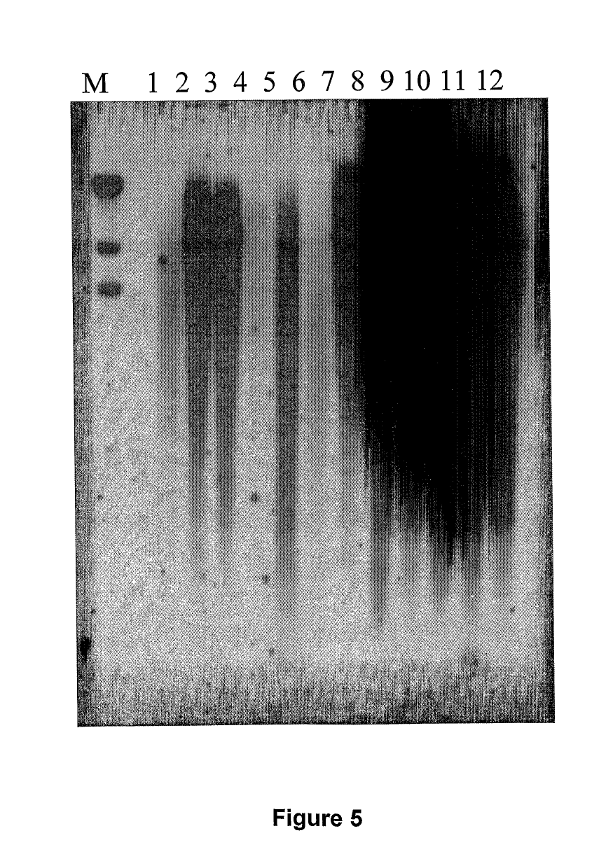

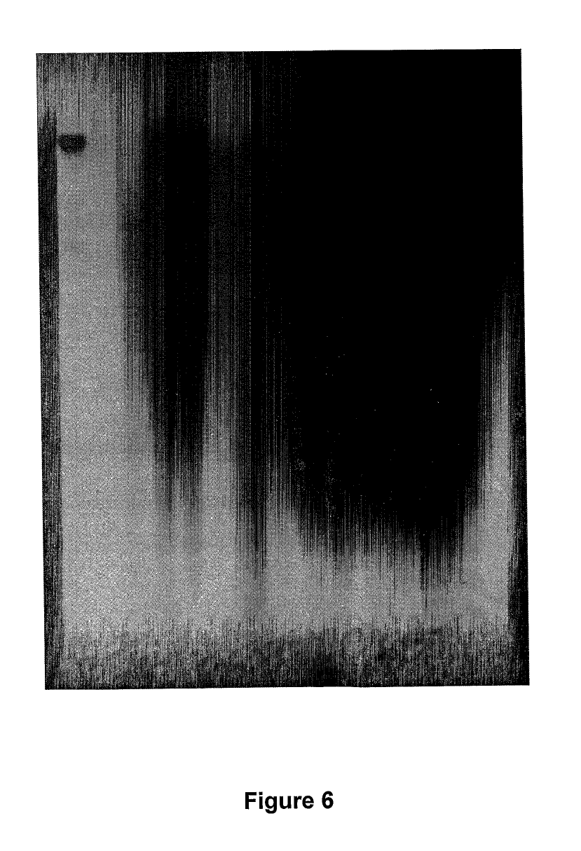

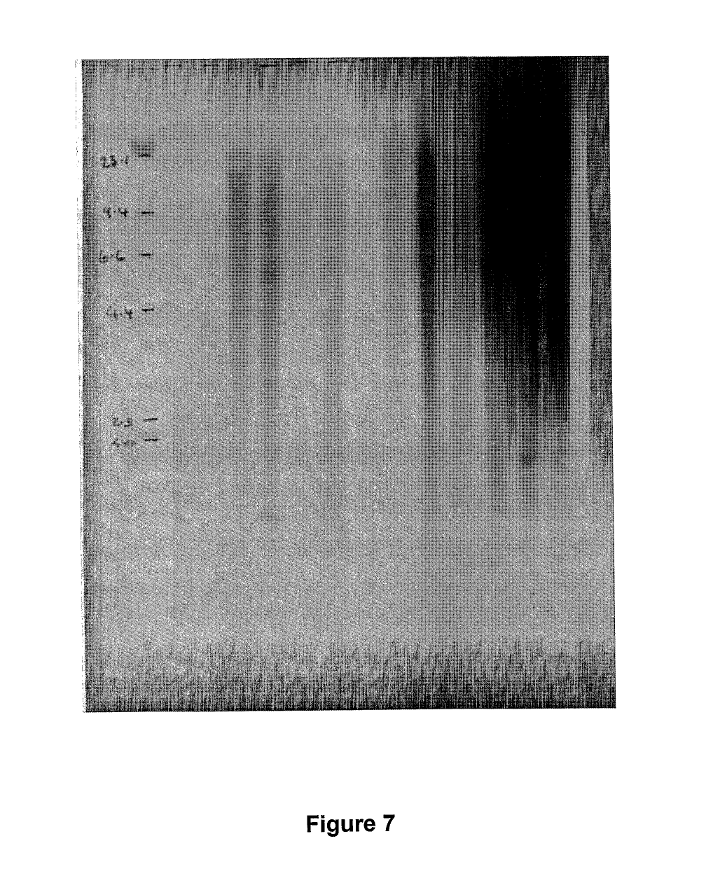

The presence of pH-modulating polypeptides such as PPM1 (SEQ ID NO 2) MAC9F1 (SEQ ID NO 4) and CAC16.5 (SEQ ID NO 6) or other sequences identified as such is correlated with the occurrence of genes encoding these proteins. It would be expected that such genes from other species would hybridize with petunia sequences such as PPM1 (SEQ ID NO 1), MAC9F1 (SEQ ID NO 3) and CAC16.5 (SEQ ID NO 5) under conditions of low stringency. As an example of this DNA was isolated from a number of floral species and subjected to Southern analysis whereby fractionated DNA was transferred to a membrane and hybridized with (i) 32P-labelled rose PPM1 (SEQ ID NO 98), FIG. 5 or (ii) 32P-labelled petunia MAC9F1 (SEQ ID NO 3) and petunia CAC16.5 (SEQ ID NO 5), FIGS. 6 and 7 respectively. Therefore the isolation of pH-modulating genes from other floral species should be possible using petunia or rose probes from genes identified as encoding pH-modulating proteins.

The isolation of pH modulating cDNAs from the plants listed above and others is accomplished by the screening of respective petal cDNA libraries with SEQ ID NO:1 and/or 3 and/or 5 using low stringency hybridisation conditions such as those described below or in the introduction of the instant specification.

Alternatively, the isolation of pH modulating cDNA fragments are accomplished using the polymerase chain reaction using primers such as those listed in the Examples above or specifically designed degenerate primers. The amplification products are cloned into bacterial plasmid vectors and DNA fragments used as probes to screen respective cDNA libraries to isolate longer and full-length pH modulating cDNA clones. The functionality and specificity of the cDNA clones are ascertained using methods described in Examples described above.

Isolation of pH Sequences from Other Species Such as Carnation, Rose, Gerbera, Chrysanthemum Etc.

The isolation of sequences that surprisingly modulate the pH of the petal vacuole without any obvious impact on other metabolic pathways (SEQ ID NO: 1 to 6) allow for the possibility of isolating similar sequences from any other species by various molecular biology and/or protein chemistry methods. These include but are not limited to preparation of cDNA libraries from RNA isolated from petal tissue, screening the petal cDNA libraries using low stringency hybridisation conditions using the labelled petunia sequences (SEQ ID NO: 1, 3 and 5) as probes, sequencing the hybridising purified cDNA clones and comparing these sequences with the petunia sequences (SEQ ID NO: 1 to 6) and searching for any sequence identity and similarity, determining expression profiles of the isolated cDNA clones and selecting those that are preferentially expressed in petals, preparing gene constructs that allow for the specific sequence to be silenced in the plant using for example, antisense expression, co-suppression or RNAi expression. Ideally the plant of interest would also be producing delphinidin (or its derivatives). This could be achieved by expressing a Flavonoid 3′, 5′ hydroxylase (F3′5′H) sequence as described in International Patent Applications PCT/AU92/00334 and/or PCT/AU96/00296 and/or PCT/JP04/11958 and/or PCT/AU03/01111.

Preparation of Petal cDNA Libraries

Total RNA is isolated from the petal tissue of flowers using the method of Turpen and Griffith (BioTechniques 4: 11-15, 1986). Poly(A)⁺ RNA is selected from the total RNA, using oligotex-dT (Trade Mark) (Qiagen) or by three cycles of oligo-dT cellulose chromatography (Aviv and Leder, Proc. Natl. Acad. Sci. USA 69: 1408, 1972).

λZAPII/Gigapack II Cloning kit (Stratagene, USA) (Short et al, Nucl. Acids Res. 16: 7583-7600, 1988) is used to construct directional petal cDNA libraries in % ZAPII using around 5 μg of poly(A)⁺ RNA isolated from petal as template.

After transfecting XL1-Blue MRF′ cells, the packaged cDNA mixtures are plated at around 50,000 pfu per 15 cm diameter plate. The plates are incubated at 37° C. for 8 hours, and the phage is eluted in 100 mM NaCl, 8 mM MgSO₄, 50 mM Tris-HCl pH 8.0, 0.01% (w/v) gelatin (Phage Storage Buffer (PSB)) (Sambrook et al, 1989, supra). Chloroform is added and the phages stored at 4° C. as amplified libraries.

Around 100,000 pfu of the amplified libraries are plated onto NZY plates (Sambrook et al, 1989, supra) at a density of around 10,000 pfu per 15 cm plate after transfecting XL1-Blue MRF′ cells, and are then incubated at 37° C. for 8 hours. After incubation at 4° C. overnight, duplicate lifts are taken onto Colony/Plaque Screen™ filters (DuPont) and are treated as recommended by the manufacturer.

### Plasmid Isolation

Helper phage R408 (Stratagene, USA) is used to excise pBluescript phagemids containing cDNA inserts from amplified λZAPII or λZAP cDNA libraries using methods described by the manufacturer.

Screening of Petal cDNA Libraries

Prior to hybridization, duplicate plaque lifts are washed in prewashing solution (50 mM Tris-HCl pH7.5, 1 M NaCl, 1 mM EDTA, 0.1% (w/v) sarcosine) at 65° C. for 30 minutes; followed by washing in 0.4 M sodium hydroxide at 65° C. for 30 minutes; then washed in a solution of 0.2 M Tris-HCl pH 8.0, 0.1×SSC, 0.1% (w/v) SDS at 65° C. for 30 minutes and finally rinsed in 2×SSC, 1.0% (w/v) SDS.

The membrane lifts from the petal cDNA libraries are hybridized with ³²P-labelled fragments of petunia PPM1 (SEQ ID NO: 1) or petunia 9F1 (SEQ ID NO: 3) or petunia CAC16.5 (SEQ ID NO: 5).

Hybridization conditions include a prehybridization step in 10% v/v formamide, 1 M NaCl, 10% w/v dextran sulphate, 1% w/v SDS at 42° C. for at least 1 hour. The ³²P-labelled fragments (each at 1×10⁶ cpm/mL) are then added to the hybridization solution and hybridization is continued at 42° C. for a further 16 hours. The filters are then washed in 2×SSC, 1% w/v SDS at 42° C. for 2×1 hour and exposed to Kodak XAR film with an intensifying screen at −70° C. for 16 hours.

Strongly hybridizing plaques are picked into PSB (Sambrook et al, 1989, supra) and rescreened to isolate purified plaques, using plating and hybridization conditions as described for the initial screening of the cDNA library. The plasmids contained in the λZAPII or λZAP bacteriophage vectors are rescued and sequence data is generated from the 3′ and 5′ ends of the cDNA inserts. New pH modulating cDNA clones are identified based on nucleic acid and predicted amino acid sequence similarity to the petunia PPM1 (SEQ ID NO: 1 and 2), MAC9F1 (SEQ ID NO: 3 and 4) or CAC16.5 (SEQ ID NO: 5 and 6)

Isolation of PPM cDNA Homologues from Rose

A Rose (cv. ‘rote rose’) petal cDNA library was constructed utilizing total RNA isolated from rose petal tissue and a λZAP cDNA synthesis kit (Stratagene) according to procedures described above and those recommended by the manufacturer. A library of 3×10⁵ pfu was thus constructed for isolation of a rose PPM1 cDNA.

The cDNA library was probed as follows. DIG-labelled petunia PPM-1 R27 cDNA fragment. Primer sets for DIG-labelling of PPM1 fragment were designed based on the petunia PPM1 sequence (Sequence ID NO: 1).

#2124: 5′-GCTAGGAGTGCTGCTGATCTTG
#2078: 5′-GGAGCCAGAAGTTTGTTATAGGAGG

The PCR conditions used for labelling of the probe were as follows,
94° C. 1 min×1
94° C. 30 sec, 55° C. 30 sec, 72° C. 1 min×25
72° C. 7 min×1

Hybond-N (Amersham) membranes were used and treated according to the manufacture's instructions. Prior to hybridization, duplicate plaques lifts were washed in a prewash solution (50 mM Tris-HCl, pH7.5, 1 M NaCl, 1 mM EDTA, 0.1% sarcosine) at 65° C. for 30 minutes. This was followed by washing in 0.4M sodium hydroxide at 65° C. for 30 minutes, then in a solution of 0.2M Tris-HCl, pH 8.0, 0.1×SSC, 0.1% (w/v) SDS at 65° C. for 30 minutes and finally rinsed in 2×SSC, 1.0% (w/v) SDS.

Hybridization conditions included a prehybridization step at 37° C. for 2-3 hr in Hybridization Buffer (5×SSC, 30% Formamide, 2% Blocking Reagent, 0.1% N-lauroylsarcosine (Sodium salt), 1% SDS, 50 mM Na-Phosphate Buffer (pH7.0)). Following removal of the prehybridization buffer hybridization mix was added which contained Hybridization Buffer (5×SSC, 30% Formamide, 2% Blocking Reagent, 0.1% N-lauroylsarcosine (Sodium salt), 1% SDS, 50 mM Na-Phosphate Buffer (pH7.0)) with DIG labelled probe added. Hybridization was carried out overnight at 37° C. Subsequent to this the filters were washed twice at 55° C. for 1 hr each.

300,000 pfu of the rose cDNA library was initially plated for screening. Two rounds of screening yielded 36 positively hybridizing clones. These were in vivo excised according to the manufacture's instructions. In each case the excised cDNA was cloned in a phagemid vector pBluescript SK− and the inserts were subsequently sequenced. Of the original 36 three clones were found to encode an identical cDNA, the longest of them, PPM1 was used for further analysis. This sequence (SEQUENCE ID NO 98) was identified as a rose PPM1 clone by reason of homology with the petunia PPM1 clone. The deduced amino acid sequence (SEQUENCE ID NO 99) when aligned with the petunia PPM1 sequence (SEQUENCE ID NO 2) also contained the same 3 amino acid residues at the C-terminus which have been identified as ‘telletale’ or typical of this class of P-ATPases.

Isolation of PPM cDNA Homologues from Carnation

Screening for a carnation PPM1 cDNA could utilize either combined rose and petunia probes or individual probes. Initially a rose PPM1 was used to screen a carnation cDNA library.

Construction of Carnation cv. Kortina Chanel cDNA Library

Twenty micrograms of total RNA was isolated from stages 1, 2 and 3 of Kortina Chanel flowers and reverse transcribed in a 50 μL volume containing 1× Superscript™ reaction buffer, 10 mM dithiothreitol (DTT), 500 μM dATP, 500 μM dGTP, 500 μM dTTP, 500 μM 5-methyl-dCTP, 2.8 μg Primer-Linker oligo from ZAP-cDNA Gigapack III Gold cloning kit (Stratagene) and 2 μL Superscript™ reverse transcriptase (BRL). The reaction mix was incubated at 37° C. for 60 minutes, then placed on ice. A ZAP-cDNA Gigapack III Gold Cloning kit (Stratagene) was used to complete the library construction. The total number of recombinants was 2.4×10⁶.

The library was subsequently titred, prior to screening for PPM1 sequences, at 1.95×10⁵ pfu (total). A 25 ml Culture of XL1 Blue MRF′ cells in 25 ml LB supplemented with 250 μl 20% Maltose and 250 μl 1M MgSO₄ was incubated until OD₆₀₀ 0.6-1. Cells were centrifuged at 4,000 rpm for 10 mins and then gently resuspended in 10 mM MgSO₄. The mixture was stored on ice. 200 μl of the XL1 Blue MRF′ cells was placed in a 12 ml falcon tube and add 10 μl of diluted library and incubated at 37° C. for 15 mins. To this was added 5 ml NZY top agar (held at 50° C.) invert gently to ensure no bubbles and pour onto small (30 ml) NZY plates pre-warmed at 42° C. These were incubated at RT to set for approximately 15 mins. Plates were inverted and incubated at 37° C. overnight to allow plaques to form.

The library was plated at 40,000 Pfu per plate over 12 large plates thus including 500,000 plaques in the primary screen. A 25 ml Culture of XL1 Blue MRF′ cells in 25 ml LB supplemented with 250 μl 20% Maltose and 250 ul 1M MgSO₄ was incubated until OD₆₀₀ 0.6-1.0 Cells were centrifuged at 4,000 rpm (approx 3,000 g) for 10 mins in an eppendorf centrifuge and then gently resuspended in 10 mM MgSO₄ and placed on ice An appropriate dilution of the library so was made to generate 40,000 pfu/10 μl per plate. Following the procedure outlined above 12 plates were generated for transfer to nylon membranes preparatory to screening for pH-modulating sequences such as PPM1, MAC9F and CAC16.5.

Following transfer the filters were transferred into prewash solution for 15 mins at 65° C. and then into denaturing solution for 15 mins at RT and then into neutralising solution for 15 mins at RT.

Filters were subjected to prehybridization (6 large per bottle) in 20 ml of 20% NEN (low stringency) at 42° C. for at least 1 hour before overnight hybridization at 42° C. with a ³²P labelled rose PPM1 DNA probe generated using PCR. Low stringency washes were carried out as follows: 6×SSC/1% SDS 55° C. for 1 hr×2, 2×SSC/1% SDS 42° C. for 40 mins, 2×SSC/1% SDS 50° C. for 20 mins and 2×SSC/1% SDS 65° C. for 30 mins. 24 putative positives were selected based on relative hybridization signal and these collected for secondary screening.

Positive “plugs” were excised and placed into an eppendorf tube containing 500 μl of PSB and 20 μl Chloroform. These were agitated for 4 hrs at room temperature, allowed to settle before removal of 1 μl into PSB for plating as before. 14 plaques were chosen for rescue and sequencing. As in the case of rose (see above) sequence analysis will reveal whether any of the clones isolated are in fact carnation PPM1 by virtue of sequence alignment and a closer examination of the C-terminal sequence of the deduced amino acid sequence derived from the cDNAs isolated as described.

### Example 7

### Use of pH Modulating Sequences

In order to modulate (increase or decrease) the petal vacuolar pH in species or cultivars of species that do not normally produce delphinidin-based pigments and do not contain a flavonoid 3′ 5′ hydroxylases capable of hydroxylating dihydroflavonols, specifically dihydrokaempferol and/or dihydroquercetin, constructs containing the combination of a F3′5′H gene (such as but not limited to F3′5′H genes described in International Patent Applications PCT/AU92/00334 and/or PCT/AU03/0111) and a pH modulating or altering sequence are introduced into a species that does not normally produce delphinidin-based pigments. Such plants may include but are not limited to rose, carnation, chrysanthemum, gerbera, orchids, Euphorbia, Begonia and apple.

In order to modulate the petal vacuolar pH in species or cultivars of species that produce delphinidin or cyanidin but have a vacuolar pH such that the color exhibited is not blue, constructs containing one or more pH modulating sequences are introduced into such species. Such plants include but are not limited to pansy, Nierembergia, lisianthus, cultivars of grapevine, lily, Kalanchoe, pelargonium, Impatiens, Catharanthus, cyclamen, Torenia, Petunia and Fuchsia.

Isolation of pH Sequences from Other Species Such as Carnation, Rose, Gerbera, Chrysanthemum Etc.

The isolation of sequences that surprisingly modulate the pH of the petal vacuole without any obvious impact on other metabolic pathways (SEQ ID NO: 1 to 6) allow for the possibility of isolating similar sequences from any other species by various molecular biology and/or protein chemistry methods. These include but are not limited to preparation of cDNA libraries from RNA isolated from petal tissue, screening the petal cDNA libraries using low stringency hybridisation conditions using the labelled petunia sequences (SEQ ID NO: 1, 3 and 5) as probes, sequencing the hybridising purified cDNA clones and comparing these sequences with the petunia sequences (SEQ ID NO: 1 to 6) and searching for any sequence identity and similarity, determining expression profiles of the isolated cDNA clones and selecting those that are preferentially expressed in petals, preparing gene constructs that allow for the specific sequence to be silenced in the plant using for example, antisense expression, co-suppression or RNAi expression. Ideally the plant of interest would also be producing delphinidin (or its derivatives). This could be achieved by expressing a Flavonoid 3′, 5′ hydroxylase (F3′5′H) sequence as described in International Patent Applications PCT/AU92/00334 and/or PCT/AU96/00296 and/or PCT/JP04/11958 and/or PCT/AU03/01111.

Preparation of Petal cDNA Libraries

Total RNA is isolated from the petal tissue of flowers using the method of Turpen and Griffith (BioTechniques 4: 11-15, 1986). Poly(A)⁺ RNA is selected from the total RNA, using oligotex-dT (Trade Mark) (Qiagen) or by three cycles of oligo-dT cellulose chromatography (Aviv and Leder, Proc. Natl. Acad. Sci. USA 69: 1408, 1972).

λZAPII/Gigapack II Cloning kit (Stratagene, USA) (Short et al, Nucl. Acids Res. 16: 7583-7600, 1988) is used to construct directional petal cDNA libraries in λZAPII using around 5 μg of poly(A)⁺ RNA isolated from petal as template.

After transfecting XL1-Blue MRF′ cells, the packaged cDNA mixtures are plated at around 50,000 pfu per 15 cm diameter plate. The plates are incubated at 37° C. for 8 hours, and the phage is eluted in 100 mM NaCl, 8 mM MgSO₄, 50 mM Tris-HCl pH 8.0, 0.01% (w/v) gelatin (Phage Storage Buffer (PSB)) (Sambrook et al, 1989, supra). Chloroform is added and the phages stored at 4° C. as amplified libraries.

Around 100,000 pfu of the amplified libraries are plated onto NZY plates (Sambrook et al, 1989, supra) at a density of around 10,000 pfu per 15 cm plate after transfecting XL1-Blue MRF′ cells, and are then incubated at 37° C. for 8 hours. After incubation at 4° C. overnight, duplicate lifts are taken onto Colony/Plaque Screen™ filters (DuPont) and are treated as recommended by the manufacturer.

### Plasmid Isolation

Helper phage R408 (Stratagene, USA) is used to excise pBluescript phagemids containing cDNA inserts from amplified λZAPII or λZAP cDNA libraries using methods described by the manufacturer.

Screening of Petal cDNA Libraries

Prior to hybridization, duplicate plaque lifts are washed in prewashing solution (50 mM Tris-HCl pH7.5, 1 M NaCl, 1 mM EDTA, 0.1% (w/v) sarcosine) at 65° C. for 30 minutes; followed by washing in 0.4 M sodium hydroxide at 65° C. for 30 minutes; then washed in a solution of 0.2 M Tris-HCl pH 8.0, 0.1×SSC, 0.1% (w/v) SDS at 65° C. for 30 minutes and finally rinsed in 2×SSC, 1.0% (w/v) SDS.

The membrane lifts from the petal cDNA libraries are hybridized with ³²P-labelled fragments of petunia PPM1 (SEQ ID NO: 1) or petunia 9F1 (SEQ ID NO: 3) or petunia CAC16.5 (SEQ ID NO: 5).

Hybridization conditions include a prehybridization step in 10% v/v formamide, 1 M NaCl, 10% w/v dextran sulphate, 1% w/v SDS at 42° C. for at least 1 hour. The ³²P-labelled fragments (each at 1×10⁶ cpm/mL) are then added to the hybridization solution and hybridization is continued at 42° C. for a further 16 hours. The filters are then washed in 2×SSC, 1% w/v SDS at 42° C. for 2×1 hour and exposed to Kodak XAR film with an intensifying screen at −70° C. for 16 hours.

Strongly hybridizing plaques are picked into PSB (Sambrook et al, 1989, supra) and rescreened to isolate purified plaques, using plating and hybridization conditions as described for the initial screening of the cDNA library. The plasmids contained in the λZAPII or λZAP bacteriophage vectors are rescued and sequence data is generated from the 3′ and 5′ ends of the cDNA inserts. New pH modulating cDNA clones are identified based on nucleic acid and predicted amino acid sequence similarity to the petunia PPM1 (SEQ ID NO: 1 and 2), MAC9F1 (SEQ ID NO: 3 and 4) or CAC16.5 (SEQ ID NO: 5 and 6)

### Construction of a Plant Transformation Vector for Down Regulation of Rose PPM1.

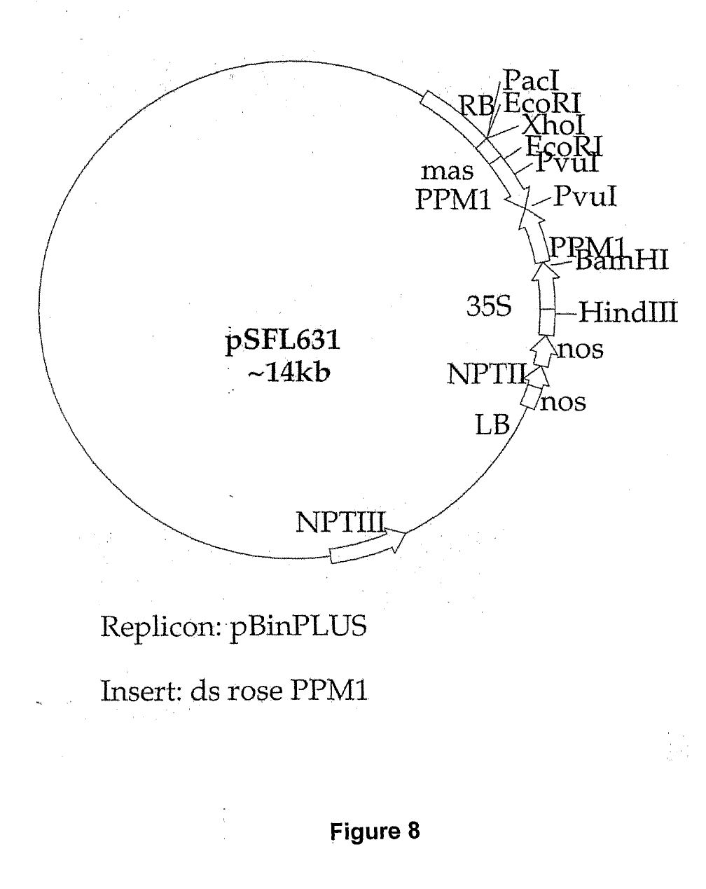

The rose PPM cDNA was used as a basis for construction of a plant transformation vector aimed at downregulation or gene knockout of rose PPM1 in rose petals. Knockout of rose PPM1 would thus lead to elevation of petal vacuolar pH and change of flower color. To achieve gene knockout a strategy aimed at production of dsRNA for rose PPM1 was used. Thus a hairpin structure was engineered using 600 bp of 5′ sequence of the cDNA and incorporated into a CaMV 35S:mas expression cassette in the binary vector pBinPLUS. This construct was named pSFL631 (FIG. 8). It was transferred into Agrobacterium tumefaciens preparatory to transformation of rose tissue according to the method described below. A further construct aimed at confining expression of the rose PPM1 knockout cassette to petal tissue is now in progress. On example of such a strategy will include the use of a rose CHS promoter. Other genes of the anthocyanin biosynthetic pathway will be a useful source of promoters for limiting expression of a gene cassette to petals as desired. Manipulation of the sequences included in further constructs will be used to alter the specificity of (i) gene knockout or silencing, and (ii) gene expression, that is expression of the pH-modulating sequences which are typically configured, using technology such as RNAi, to downregulate or silence the target gene. Such pH-modulating sequences will include PPM1, MAC9F1 and CAC16.5 homologues from rose.

### Construction of a Plant Transformation Vector for Down Regulation of Carnation PPM1.

The carnation PPM cDNA will be used as a basis for construction of a plant transformation vectors aimed at down regulation or gene knockout of carnation PPM1 in carnation petals. Knockout of carnation PPM1 would thus lead to elevation of petal vacuolar pH and change in flower color. To achieve gene knockout a strategy aimed at production of dsRNA for carnation PPM1 is to be used. Thus a hairpin structure will be engineered using sequence of the cDNA from a region specific to the PPM1 sequence and incorporated into both (i) constitutive, and (ii) petal-specific gene expression cassettes. In the former a CaMV 35S expression cassette (CaMV 35S promoter and terminator elements) and in the latter a petal specific promoter from carnation. A promoter from a carnation ANS gene is one example of a promoter for petal-specific expression which could be engineered. The anthocyanin pathway genes provide a useful source of promoters for controlling petal-specific gene expression. However, such expression is not confined to the use of these promoters. dsRNA (RNAi) gene silencing constructs are based on a 500 bp inverted repeat with an intervening 182 bp intron all under the control of 35S promoter or a petal specific promoter such as that from a carnation ANS gene.

### Carnation PPM1-ANS Intermediate

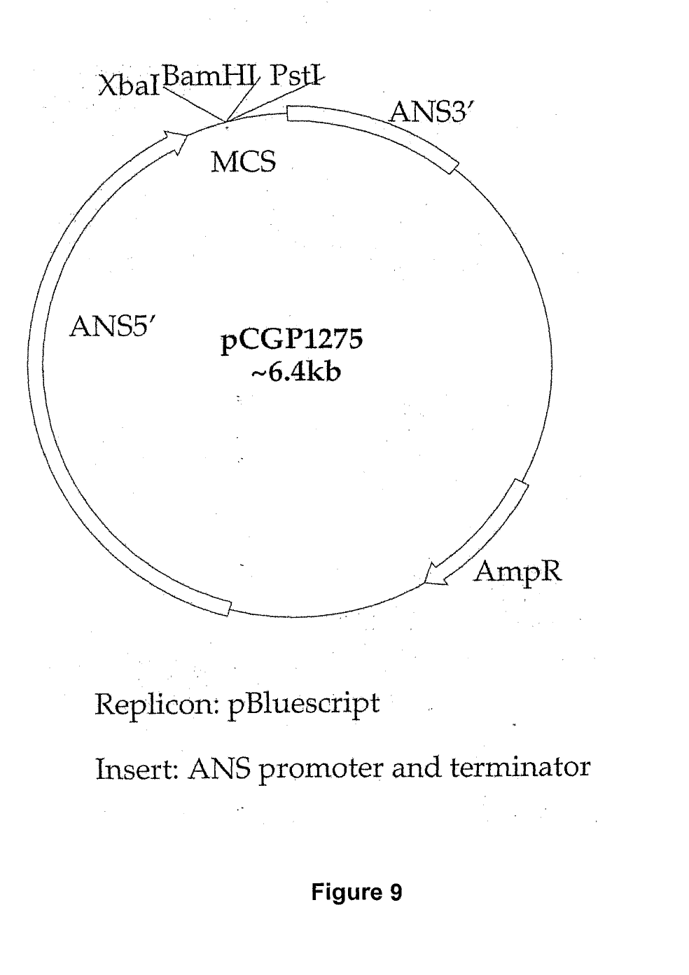

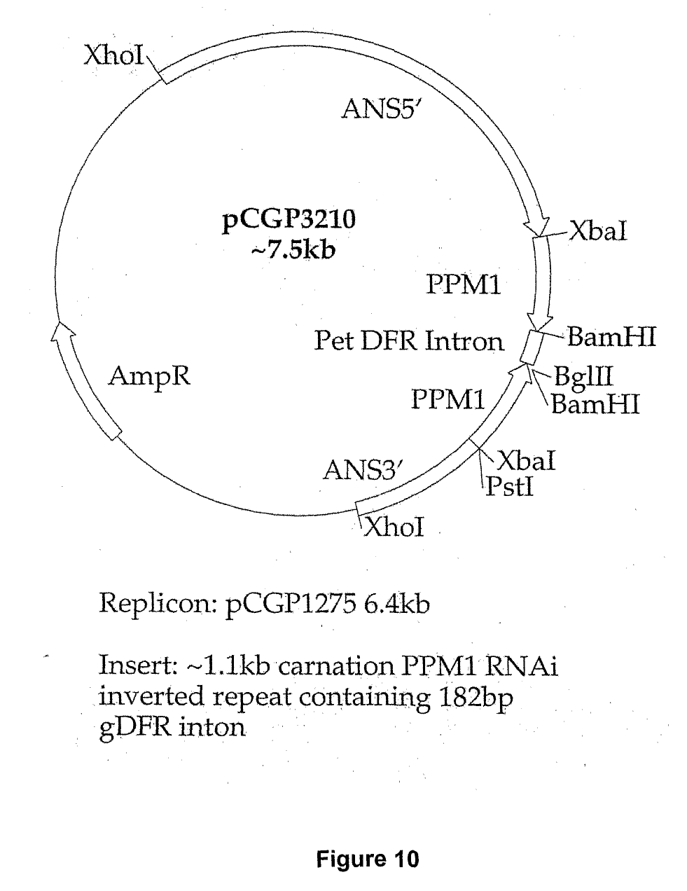

The intron will be cloned into pCGP1275 (FIG. 9) using BamHI creating pCGP1275i. The sense carnation PPMI (carnPPM1) will then be cloned into pCGP1275i using XbaI/BamHI creating pCGP12751-s-carnPPM1. The antisense PPM1 will then be cloned into pCGP1275i-s-carnPPM1 using PstI/XbaI creating pCGP3210 (FIG. 10).

Carnation PPM1-ANS in pWTT2132 Binary Transformation Vector

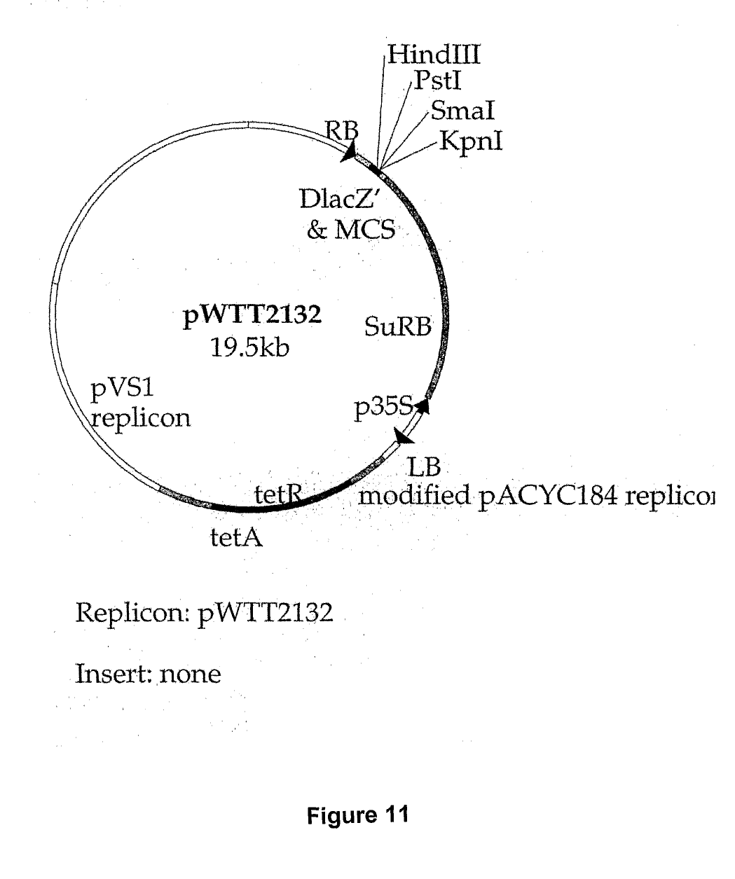

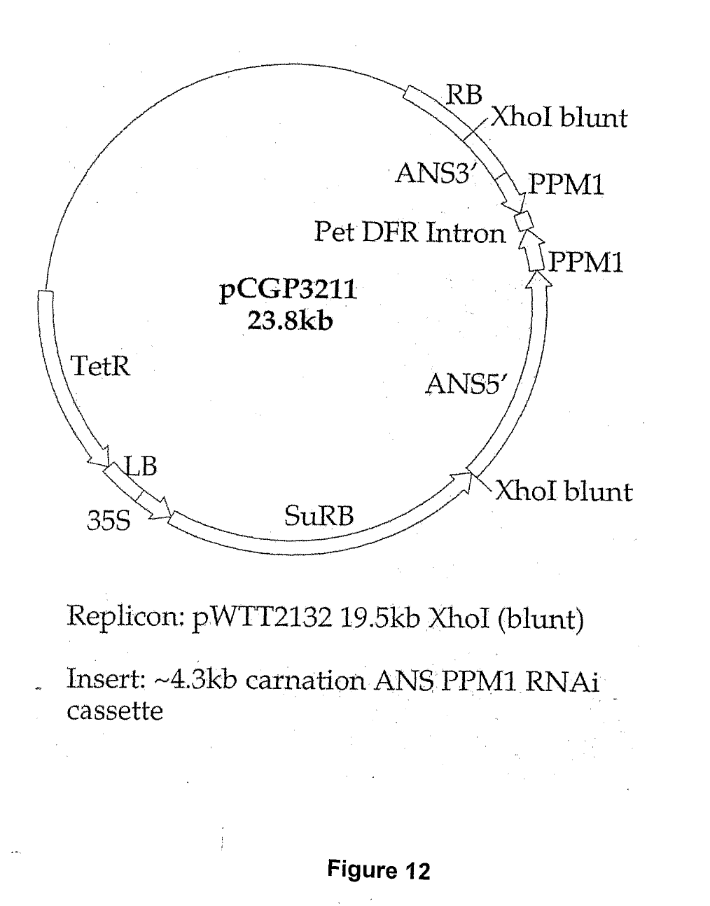

The carnPPM1/ANS cassette will then be cut out of pCGP3210 with XhoI (blunt) to be ligated into the binary transformation vector pWTT2132 (FIG. 11) to create the binary transformation vector pCGP3211 (FIG. 12)

Carnation PPM1-ANS in pBinPLUS Binary

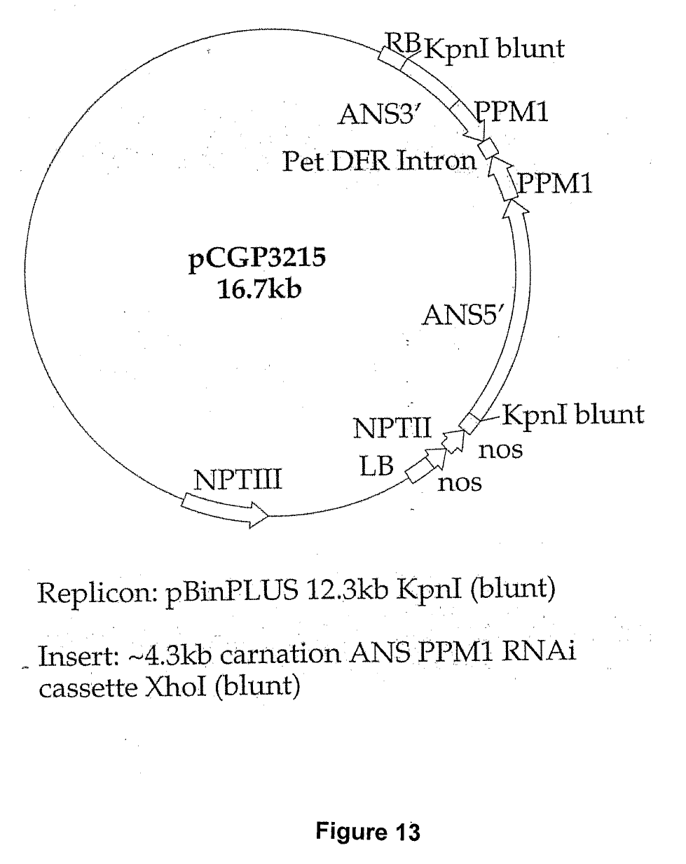

The carnPPM1/ANS cassette will be again cut out of pCGP3210 XhoI (blunt) and ligated into pBinPLUS KpnI (blunt) to create the binary transformation vector pCGP3215 (FIG. 13).

Carnation PPM1-ANS in pCGP2355 Binary

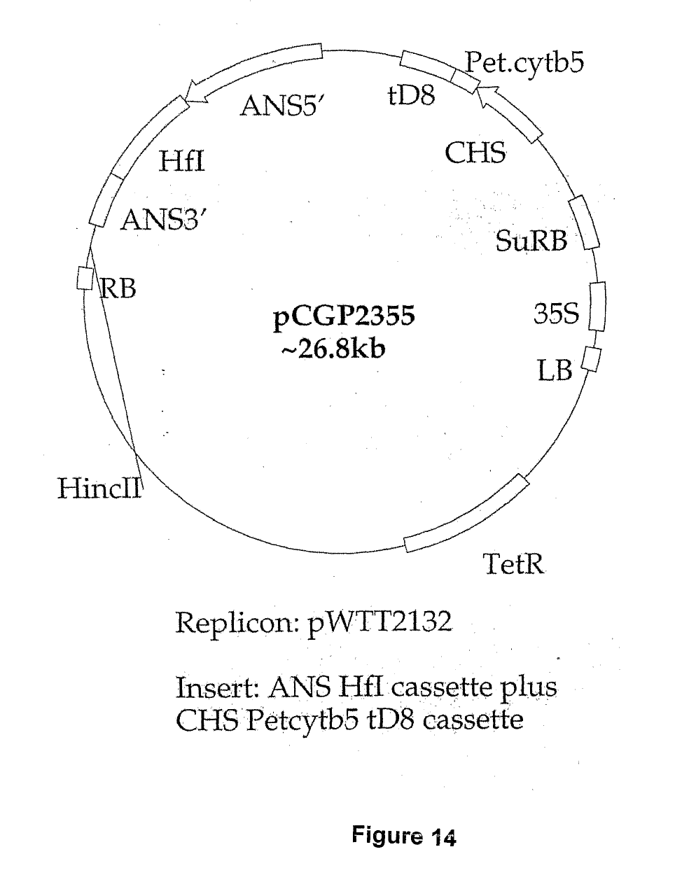

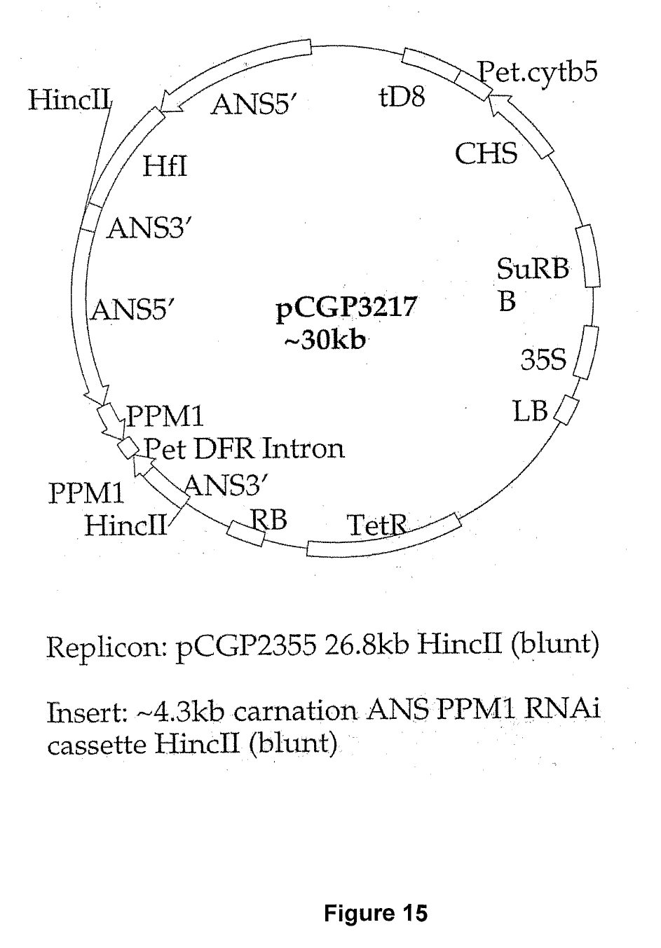

The carnPPM1/ANS cassette will again be cut out of pCGP3210 with to be ligated into pCGP2355 (FIG. 14) to create the binary transformation vector pCGP3217 (FIG. 15)

### PPM1-35S Intermediate

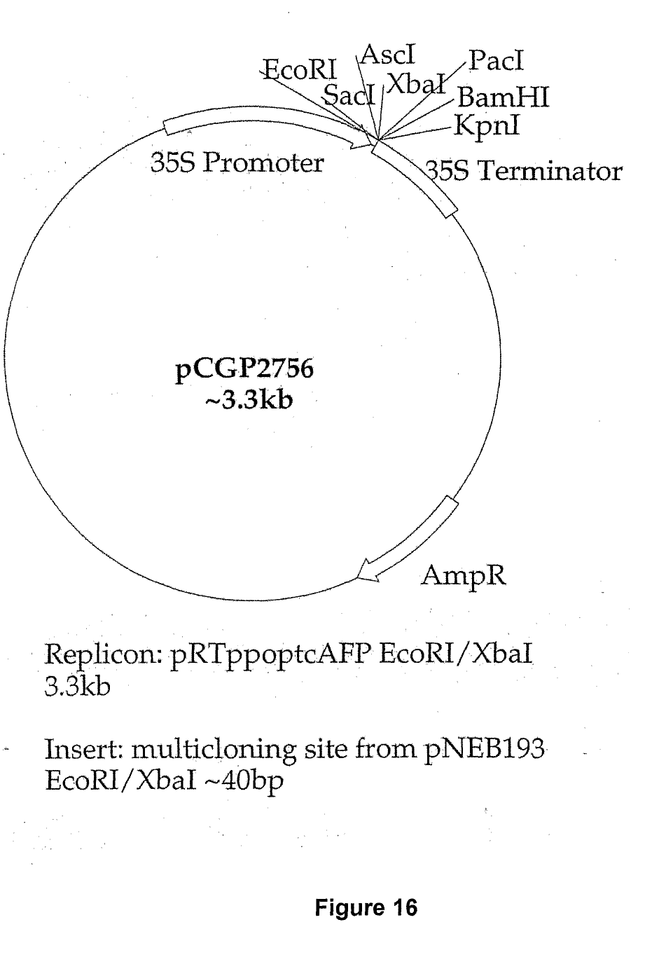

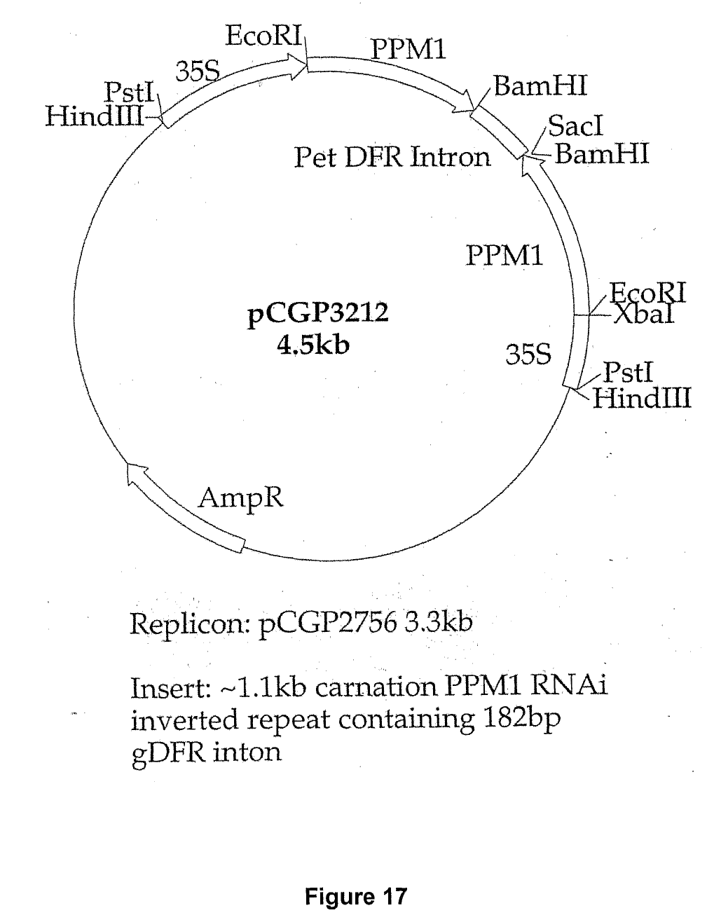

The carnation ANS intron will also be cloned into pCGP2756 (FIG. 16) using BamHI creating pCGP2756i. The sense carnPPMI will then be cloned into pCGP2756i using EcoRI/BamHI creating pCGP2756i-s-carnPPM1. The antisense PPM1 will then be cloned into pCGP2756i-s-carnPPM1 using SacI/XbaI creating pCGP3212 (FIG. 17).

Carnation PPM1-35S in pWTT2132 Binary

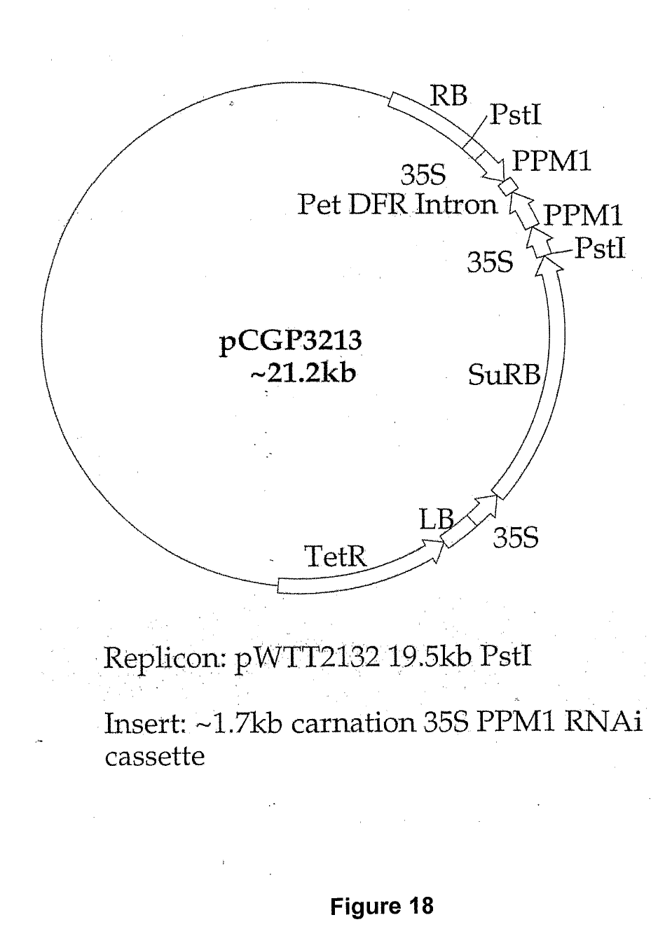

The carnPPM1/ANS cassette will then be cut out of pCGP3212 with PstI to be ligated into pWTT2132 to create the binary transformation vector pCGP3213 (FIG. 18)

Carnation PPM1-35S in pBinPLUS Binary

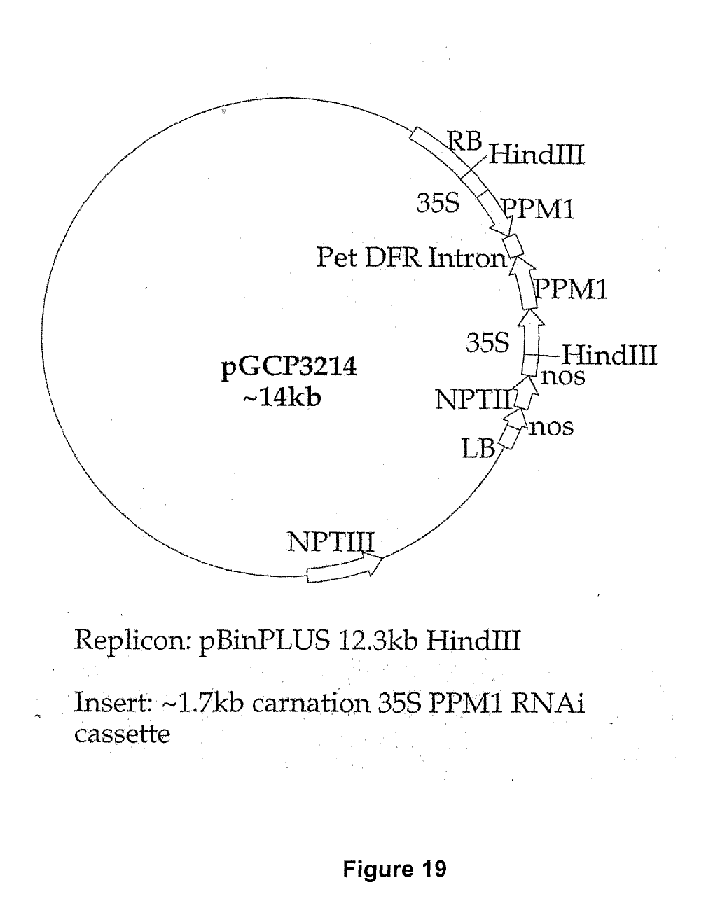

The carnPPM1/ANS cassette will then be cut out of pCGP3212 with HindIII to be ligated into pWTT2132 to create the binary transformation vector pCGP3214 (FIG. 19).

Carnation PPM1-35S in pCGP2355 Binary

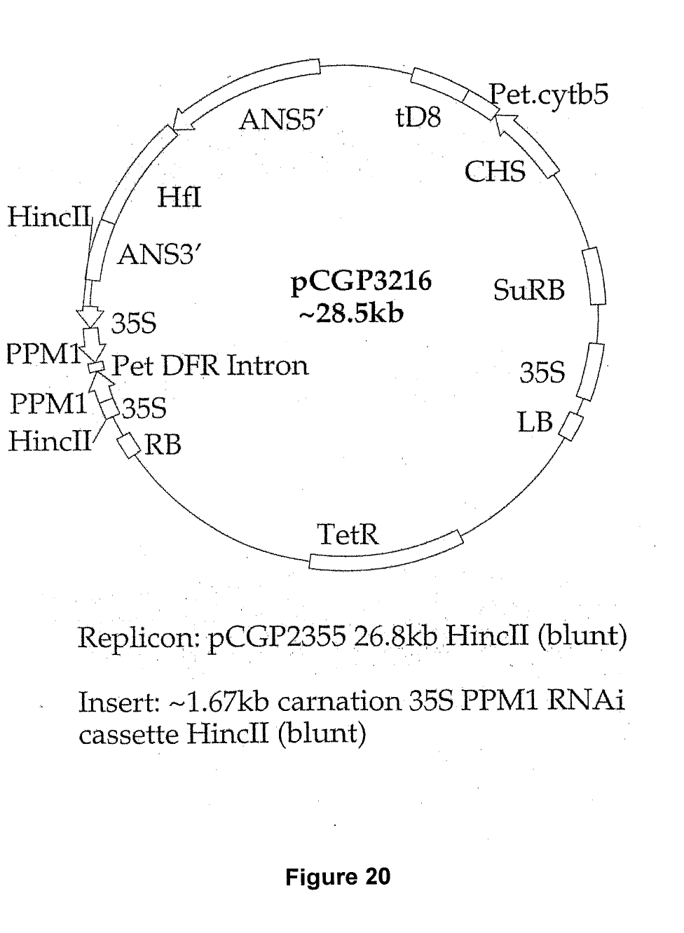

The carnPPM1/ANS cassette will be cut out of pCGP3212 with HindIII to be ligated into pCGP2355 to create the binary transformation vector pCGP3216 (FIG. 20).

The transformation vectors generated above are to be used to engineer pH-modulation in a number of different targets and tissues. In general expression of pH-modulating sequences, such as silencing of carnation PPM1, will be either constitutive or petal-specific. Targets for transformation will include both carnations which produce delphindin and those that do not. In each case assessment of the efficacy of pH modulation will be measured through measurement of pH and/or visualisation of color change.

Construction of plant transformation vectors for down regulation of pH modulating genes. It is envisaged that the above strategy would be used to downregulate or silence pH modulating genes such as PPM1, MAC9F1 and CAC16.5 and their homologues in carnation, rose, gerbera, chrysanthemum and other floral species of commercial value. Typically such a strategy would involve isolation of a homologue from the target species. However, the strategy is not confined to this approach as gene silencing technologies such as RNAi can be applied across species given conservation of appropriate sequences. Determination of whether such a strategy would be effective across species could best be arrived at through the isolation and characterisation of homologues form a target species however. Such characterisation would include determination of the nucleotide sequence and subsequently the deduced amino acid sequence of pH-modulating genes such as PPM1, MAC9F1 and CAC16.5. It is thus conceivable that a rose PPM1 sequence could be used to design effective pH-modulating gene silencing constructs for use in another species such as carnation, gerbera or chrysanthemum.

Binary transformation vectors, such as those described above, are used in plant transformation experiments to generate plants carrying the desired genes, in this case pH-modulating genes. It is in this fashion that it is intended to use pH-modulating genes from petunia, rose and carnation to alter petal pH and thus flower color in rose, carnation, gerbera, chrysanthemum and other floral species of commercial value.

### Plant Transformations

Rosa hybrida Transformations

Introduction of pH modulating sequences into roses is achieved using methods as described in U.S. Pat. No. 542,841 (PCT/US91/04412) or Robinson and Firoozabady (Scientia Horticulturae, 55: 83-99, 1993) or Rout et al. (Scientia Horticulturae, 81: 201-238, 1999) or Marchant et al. (Molecular Breeding 4: 187-194, 1998) or Li et al (Plant Physiol Biochem., 40, 453-459, 2002) or Kim et al (Plant Cell Tissue and Organ Culture, 78, 107-111, 2004) or by any other method well known in the art.

Dianthus caryophyllus Transformations

Introduction of pH modulating sequences into carnations is achieved using methods as described in International Patent Application No. PCT/US92/02612, or International Patent Application No. PCT/AU96/00296, Lu et al. (Bio/Technology 9: 864-868, 1991), Robinson and Firoozabady (1993, supra) or by any other method well known in the art.

### Chrysanthemum Transformations

Introduction of pH modulating sequences into chrysanthemum is achieved using methods as described in da Silva (Biotechnology Advances, 21, 715-766, 2003) or Aswath et al (Plant Science 166, 847-854, 2004) or Aida et al (Breeding Sci. 54, 51-58, 2004) or by any other method well known in the art.

### Gerbera Transformations

Introduction of pH modulating sequences into gerbera is achieved using methods as described in Elomaa and Teeri (In YPS Bajaj, ed, Biotechnology in Agriculture and Forestry, Transgenic Crops III, Springer-Verlag, Berlin, 48, 139-154, 2001) or by any other method well known in the art.

### Ornamental Plant Transformations

Introduction of pH modulating sequences into ornamental plants is achieved using methods as described or reviewed in Deroles et al (In: Geneve R L, Preece J E & Markle S A (eds) Biotechnology of Ornamental Plants CAB International, Wallingford 87-119, 1997) or Tanaka et al (In: Chopra V L, Malik V S & Bhat S R (eds) Applied Plant Biotechnology. Oxford & IBH, New Delhi, 177-231, 1999) or Tanaka et al (Plant Cell, Tissue and Organ Culture 80, 1-24, 2005) by any other method well known in the art.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

### BIBLIOGRAPHY

Altschul et al, Nucl. Acids Res. 25: 3389-3402, 1997
Arango et al, Planta, 216:335-365, 2003
Ausubel et al., “Current Protocols in Molecular Biology” John Wiley & Sons Inc, 1994-1998, Chapter 15.
Baxter et al, PNAS, 102:2649-2654, 2005
Bonner and Laskey, Eur. J. Biochem. 46: 83, 1974.
Brugliera et al, Plant J. 5, 81-92, 1994
de Vetten et al, Genes Dev. 11: 1422-1434, 1997
de Vetten et al, Plant Cell 11(8):1433-1444, 1999
de Vlaming et al Theor. Appl. Genet. 66, 271-278, 1983
Di Sansebastiano et al, Plant Physiology, 126, 78-86, 2001
Frohman et al, PNAS 85: 8998-9002, 1988
Fukada-Tanaka et al Nature 407, 581, 2000
Holton et al, Nature 366: 276-279, 1993
Holton and Cornish, Plant Cell 7: 1071-1083, 1995
Jahn et al JBC, 277, 6353-6358, 2002
Koes et al, Trends in Plant Science, May 2005
Logemann et al, Anal Biochem. 163(1):16-20, 1987
Merrifield, J. Am. Chem. Soc. 85:2149, 1964
Mol et al, Trends Plant Sci. 3: 212-217, 1998
Plant Molecular Biology Labfax, Croy (ed), Bios scientific Publishers, Oxford, UK, 1993
Plant Molecular Biology Manual (2^{nd} edition), Gelvin and Schilperoot (eds), Kluwer Academic Publisher, The Netherlands, 1994
Quattrocchio et al, Plant J. 13, 475-488, 1998
Quattrocchio et al, Plant Cell, 2005 (submitted)
Sambrook et al, Molecular Cloning: A Laboratory Manual, 2^{nd} edition, Cold Spring Harbor Laboratories, Cold Spring Harbor, N.Y., USA, 1989
Sambrook and Russell, Molecular Cloning: A Laboratory Manual 3^{rd} edition, Cold Spring Harbor Laboratories, Cold Spring Harbor, N.Y., USA, 2001
Spelt, C. et al. Plant Cell 14, 2121-2135, 2002
Tanaka et al, Plant Cell, Tissue and Organ Culture 80 (1): 1-24, 2005
van Houwelingen et al, Plant J. 13(1). 39-50, 1998
Verdonk et al. Phytochemistry 62:997-1008, 2003.
Winkel-Shirley, Plant Physiol. 126: 485-493, 2001a
Winkel-Shirley, Plant Physiol. 127: 1399-1404, 2001b
Yoshida et al. Nature 373, 291, 1995

1. An isolated nucleic acid molecule comprising a sequence of nucleotides encoding or complementary to a sequence encoding a pH modulating or altering gene or a polypeptide having pH modulating or altering activity wherein expression of said nucleic acid molecule alters or modulates pH inside the cell or vacuole.

2. The isolated nucleic acid molecule of claim 1 comprising a nucleotide sequence substantially as set forth in SEQ ID NO:1, 3 or 5 or a nucleotide sequence having at least about 50% identity thereto or capable of hybridizing to the nucleotide sequence set forth in SEQ ID NO:1, 3 or 5 under low stringency conditions.

3. The isolated nucleic acid molecule of claim 2 comprising the nucleotide sequence set forth in SEQ ID NO:1.

4. The isolated nucleic acid molecule of claim 2 comprising the nucleotide sequence set forth in SEQ ID NO:3.

5. The isolated nucleic acid molecule of claim 2 comprising the nucleotide sequence set forth in SEQ ID NO:5.

6. The isolated nucleic acid molecule of claim 1 encoding an amino acid sequence set forth in SEQ ID NO:2, 4 or 6 or an amino acid sequence having at least 50% similarity thereto or a truncated form of SEQ ID NO:2, 4 or 6.

7. The isolated nucleic acid molecule of claim 6 encoding the amino acid sequence set forth in SEQ ID NO:2.

8. The isolated nucleic acid molecule of claim 6 encoding the amino acid sequence set forth in SEQ ID NO:4.

9. The isolated nucleic acid molecule of claim 6 encoding the amino acid sequence set forth in SEQ ID NO:6.

10. The isolated nucleic acid molecule of any one of claims 1 to 9 fused to or otherwise associated with a gene encoding enzyme of the anthocyanin pathway.

11. A genetic construct comprising a nucleic acid molecule operably linked to a promoter such that upon expression a mRNA transcript is produced which is antisense to the nucleic acid molecule of any one of claims 1 to 9.

12. A genetic construct comprising a nucleic acid molecule operably linked to a promoter such that upon expression a mRNA transcript is produced which is sense to the nucleic acid molecule of any one of claims 1 to 9.

13. A method for modulating the pH in a vacuole of a plant cell said method comprising introducing into said plant cell or a parent or relative of said plant cell a genetic construct of claim 11 or 12 and culturing the plant cell or plant comprising said cell or parent or relative of said cell under conditions to permit expression of the nucleic acid molecule in the genetic construct.

14. The method of claim 13 wherein the plant or plant cell is or is from a plant selectively from Dianthus spp, Rosa spp, Chrysanthemum spp, Cyclamen spp, Iris spp, Pelargonium spp, Liparieae, Geranium spp, Saintpaulic spp and Plumbago spp.

15. A method for producing a transgenic flowering plant capable of synthesizing a pH modulating or altering protein, said method comprising stably transforming a cell of a suitable plant with a nucleic acid sequence which comprises a sequence of nucleotides encoding said pH modulating or altering proteins under conditions permitting the eventual expression of said nucleic acid sequence, regenerating a transgenic plant from the cell and growing said transgenic plant for a time and under conditions sufficient to permit the expression of the nucleic acid sequence.

16. The method of claim 15 wherein the nucleic acid sequence is substantially as set forth in SEQ ID NO:1, 3 or 5 or a nucleotide sequence having at least about 50% identity thereto or capable of hybridizing to the nucleotide sequence set forth in SEQ ID NO:1, 3 or under low stringency conditions.

17. The method of claim 16 wherein the nucleic acid sequence comprises the nucleotide sequence set forth in SEQ ID NO:1.

18. The method of claim 16 wherein the nucleic acid sequence comprises the nucleotide sequence set forth in SEQ ID NO:3.

19. The method of claim 16 wherein the nucleic acid sequence comprises the nucleotide sequence set forth in SEQ ID NO:5.

20. The method of claim 15 wherein the nucleic acid sequence encodes an amino acid sequence set forth in SEQ ID NO:2, 4 or 6 or an amino acid sequence having at least 50% similarity thereto or a truncated form of Seq ID NO:2, 4 or 6.

21. The method of claim 20 wherein the nucleic acid sequence encodes the amino acid sequence set forth in SEQ ID NO:2.

22. The method of claim 20 wherein the nucleic acid sequence encodes the amino acid sequence set forth in SEQ ID NO:4.

23. The method of claim 20 wherein the nucleic acid sequence encodes the amino acid sequence set forth in SEQ ID NO:6.

24. The method of claim 16 wherein the plant or plant cell is or is from a plant selectively from Dianthus spp, Rosa spp, Chrysanthemum spp, Cyclamen spp, Iris spp, Pelargonium spp, Liparieae, Geranium spp, Saintpaulic spp and Plumbago spp.

25. A method for producing a transgenic plant with reduced indigenous or existing pH modulating or altering activity, said method comprising stably transforming a cell of a suitable plant with a nucleic acid molecule which comprises a sequence of nucleotides encoding or complementary to a sequence encoding a pH modulating activity, regenerating a transgenic plant from the cell and where necessary growing said transgenic plant under conditions sufficient to permit the expression of the nucleic acid.

26. A method for producing a genetically modified plant with reduced indigenous or existing pH modulating or altering protein activity, said method comprising altering the pH modulating or altering nucleic acid molecule through modification of the indigenous sequences via homologous recombination from an appropriately altered pH modulating or altering gene or derivative or part thereof introduced into the plant cell, and regenerating the genetically modified plant from the cell.

27. The method of claim 25 or 26 comprising a nucleotide sequence substantially as set forth in SEQ ID NO:1, 3 or 5 or a nucleotide sequence having at least about 50% identity thereto or capable of hybridizing to the nucleotide sequence set forth in SEQ ID NO:1, 3 or 5 under low stringency conditions.

28. The method of claim 25 or 26 wherein the nucleic acid sequence comprises the nucleotide sequence set forth in SEQ ID NO:1.

29. The method of claim 25 or 26 wherein the nucleic acid sequence comprises the nucleotide sequence set forth in SEQ ID NO:3.

30. The method of claim 25 or 26 wherein the nucleic acid sequence comprises the nucleotide sequence set forth in SEQ ID NO:5.

31. The method of claim 25 or 26 wherein the nucleic acid sequence encodes an amino acid sequence set forth in SEQ ID NO:2, 4 or 6 or an amino acid sequence having at least 50% similarity thereto or a truncated form of Seq ID NO:2, 4 or 6.

32. The wherein the nucleic acid sequence comprises claim 31 wherein the nucleic acid sequence encodes the amino acid sequence set forth in SEQ ID NO:2.

33. The method of claim 31 wherein the nucleic acid sequence encodes the amino acid sequence set forth in SEQ ID NO:4.

34. The method of claim 31 wherein the nucleic acid sequence encodes the amino acid sequence set forth in SEQ ID NO:6.

35. The method of claim 25 or 26 wherein the plant or plant cell is or is from a plant selectively from Dianthus spp, Rosa spp, Chrysanthemum spp, Cyclamen spp, Iris spp, Pelargonium spp, Liparieae, Geranium spp, Saintpaulic spp and Plumbago spp.

36. A method for producing a transgenic plant capable of expressing a recombinant gene encoding a pH modulating or altering protein or part thereof or which carries a nucleic acid sequence which is substantially complementary to all or a part of a mRNA molecule encoding a pH modulating or altering protein, said method comprising stably transforming a cell of a suitable plant with the isolated nucleic acid molecule comprising a sequence of nucleotides encoding, or complementary to a sequence encoding, a pH modulating or altering protein, where necessary under conditions permitting the eventual expression of said isolated nucleic acid molecule, and regenerating a transgenic plant from the cell.

37. The method of claim 36 comprising a nucleotide sequence substantially as set forth in SEQ ID NO:1, 3 or 5 or a nucleotide sequence having at least about 50% identity thereto or capable of hybridizing to the nucleotide sequence set forth in SEQ ID NO:1, 3 or 5 under low stringency conditions.

38. The method of claim 37 wherein the nucleic acid sequence comprises the nucleotide sequence set forth in SEQ ID NO:1.

39. The method of claim 37 wherein the nucleic acid sequence comprises the nucleotide sequence set forth in SEQ ID NO:3.

40. The method of claim 37 wherein the nucleic acid sequence comprises the nucleotide sequence set forth in SEQ ID NO:5.

41. The method of claim 36 wherein the nucleic acid sequence encodes an amino acid sequence set forth in SEQ ID NO:2, 4 or 6 or an amino acid sequence having at least 50% similarity thereto or a truncated form of Seq ID NO:2, 4 or 6.

42. The method of claim 41 wherein the nucleic acid sequence encodes the amino acid sequence set forth in SEQ ID NO:2.

43. The method of claim 41 wherein the nucleic acid sequence encodes the amino acid sequence set forth in SEQ ID NO:4.

44. The method of claim 41 wherein the nucleic acid sequence encodes the amino acid sequence set forth in SEQ ID NO:6.

45. The method of claim 36 wherein the plant or plant cell is or is from a plant selectively from Dianthus spp, Rosa spp, Chrysanthemum spp, Cyclamen spp, Iris spp, Pelargonium spp, Liparieae, Geranium spp, Saintpaulic spp and Plumbago spp.

47. An isolated cell, plant or part of a genetically modified plant or progeny thereof which cell, plant or part comprises an altered pH in a vacuole of the cell or cells of the plant or plant parts.

48. The plant part of claim 47 selected from a flower, fruit, vegetable, nut, root, stem, leaf or seed.

49. The use of the genetic sequences described herein in the manufacture of a genetic construct capable of expressing a pH modulating or altering protein or down-regulating an indigenous pH modulating protein in a plant.

50. An isolated nucleic acid molecule comprising the nucleotide sequence set forth in SEQ ID NO:98.

51. An isolated nucleic acid molecule comprising the nucleotide sequence which encodes the amino acid sequence set forth in SEQ ID NO:99:

52. An isolated protein comprising the nucleotide sequence set forth in SEQ ID NO:99.
